SEQUENCE LISTING (90 sequences)

SEQ ID NO: 1 - DNA, 18 bp, Homo sapiens
  source 1..18: mol_type = genomic DNA
aaccaccaccgtgaccct

SEQ ID NO: 2 - DNA, 20 bp, Homo sapiens
  source 1..20: mol_type = genomic DNA
aacccactcccatgataact

SEQ ID NO: 3 - DNA, 17 bp, Homo sapiens
  source 1..17: mol_type = genomic DNA
accaccagggctcagtc

SEQ ID NO: 4 - DNA, 16 bp, Homo sapiens
  source 1..16: mol_type = genomic DNA
acaggagagatggagg

SEQ ID NO: 5 - DNA, 12 bp, Homo sapiens
  source 1..12: mol_type = genomic DNA
aactatatatat

SEQ ID NO: 6 - DNA, 18 bp, Homo sapiens
  source 1..18: mol_type = genomic DNA
acatatatacctatatat

SEQ ID NO: 7 - DNA, 17 bp, Homo sapiens
  source 1..17: mol_type = genomic DNA
actggagccccctgagg

SEQ ID NO: 8 - DNA, 15 bp, Homo sapiens
  source 1..15: mol_type = genomic DNA
aacaccagcgtcatg

SEQ ID NO: 9 - DNA, 20 bp, Homo sapiens
  source 1..20: mol_type = genomic DNA
acagagctgatccatgcccc

SEQ ID NO: 10 - DNA, 19 bp, Homo sapiens
  source 1..19: mol_type = genomic DNA
accatcctcagctcactcc

SEQ ID NO: 11 - DNA, 12 bp, Homo sapiens
  source 1..12: mol_type = genomic DNA
accatcatcatc

SEQ ID NO: 12 - DNA, 11 bp, Homo sapiens
  source 1..11: mol_type = genomic DNA
agaggggatgg

SEQ ID NO: 13 - DNA, 19 bp, Homo sapiens
  source 1..19: mol_type = genomic DNA
acccatcacgcccacctgg

SEQ ID NO: 14 - DNA, 20 bp, Homo sapiens
  source 1..20: mol_type = genomic DNA
aaggagatggaggctcagag

SEQ ID NO: 15 - DNA, 18 bp, Homo sapiens
  source 1..18: mol_type = genomic DNA
aaagagatatatatatct

SEQ ID NO: 16 - DNA, 20 bp, Homo sapiens
  source 1..20: mol_type = genomic DNA
aagggtggatgggtggatgg

SEQ ID NO: 17 - DNA, 19 bp, Homo sapiens
  source 1..19: mol_type = genomic DNA
accgggcctcactcactgc

SEQ ID NO: 18 - DNA, 20 bp, Homo sapiens
  source 1..20: mol_type = genomic DNA
acacacctgggctcccatgc

SEQ ID NO: 19 - DNA, 17 bp, Homo sapiens
  source 1..17: mol_type = genomic DNA
aactccacagaggaccc

SEQ ID NO: 20 - DNA, 15 bp, Homo sapiens
  source 1..15: mol_type = genomic DNA
aaggtgagtgagtgg

SEQ ID NO: 21 - DNA, 16 bp, Homo sapiens
  source 1..16: mol_type = genomic DNA
aacatataatatatat

SEQ ID NO: 22 - DNA, 18 bp, Homo sapiens
  source 1..18: mol_type = genomic DNA
aaacctcgcatcctatct

SEQ ID NO: 23 - DNA, 20 bp, Homo sapiens
  source 1..20: mol_type = genomic DNA
accagggctcagtgatcagg

SEQ ID NO: 24 - DNA, 20 bp, Homo sapiens
  source 1..20: mol_type = genomic DNA
aatggatggatggatggatg

SEQ ID NO: 25 - DNA, 17 bp, Homo sapiens
  source 1..17: mol_type = genomic DNA
acacacctccacagggt

SEQ ID NO: 26 - DNA, 20 bp, Homo sapiens
  source 1..20: mol_type = genomic DNA
aaggagtattccctcaggtc

SEQ ID NO: 27 - DNA, 13 bp, Homo sapiens
  source 1..13: mol_type = genomic DNA
aaccctacctact

SEQ ID NO: 28 - DNA, 18 bp, Homo sapiens
  source 1..18: mol_type = genomic DNA
acagccctggctactagc

SEQ ID NO: 29 - DNA, 11 bp, Homo sapiens
  source 1..11: mol_type = genomic DNA
agctcatcccc

SEQ ID NO: 30 - DNA, 18 bp, Homo sapiens
  source 1..18: mol_type = genomic DNA
aaggatcgtgcagcgagg

SEQ ID NO: 31 - DNA, 16 bp, Homo sapiens
  source 1..16: mol_type = genomic DNA
aaatttatatacatat

SEQ ID NO: 32 - DNA, 17 bp, Homo sapiens
  source 1..17: mol_type = genomic DNA
aaagtggagggaggatg

SEQ ID NO: 33 - DNA, 13 bp, Homo sapiens
  source 1..13: mol_type = genomic DNA
accctatatatat

SEQ ID NO: 34 - DNA, 19 bp, Homo sapiens
  source 1..19: mol_type = genomic DNA
aatataatataatatatat

SEQ ID NO: 35 - DNA, 17 bp, Homo sapiens
  source 1..17: mol_type = genomic DNA
acgccagtctctgcccc

SEQ ID NO: 36 - DNA, 20 bp, Homo sapiens
  source 1..20: mol_type = genomic DNA
accatccccctcagtgagtc

SEQ ID NO: 37 - DNA, 15 bp, Homo sapiens
  source 1..15: mol_type = genomic DNA
acagagtcccagagc

SEQ ID NO: 38 - DNA, 20 bp, Homo sapiens
  source 1..20: mol_type = genomic DNA
actcactccctcccctcctc

SEQ ID NO: 39 - DNA, 20 bp, Homo sapiens
  source 1..20: mol_type = genomic DNA
acccaggagatgcagagagt

SEQ ID NO: 40 - DNA, 20 bp, Homo sapiens
  source 1..20: mol_type = genomic DNA
aaattacaccgcagcctcgg

SEQ ID NO: 41 - DNA, 16 bp, Homo sapiens
  source 1..16: mol_type = genomic DNA
acccatccatccatcc

SEQ ID NO: 42 - DNA, 18 bp, Homo sapiens
  source 1..18: mol_type = genomic DNA
aaccacatccacatccac

SEQ ID NO: 43 - DNA, 20 bp, Homo sapiens
  source 1..20: mol_type = genomic DNA
accccccccccccccccccc

SEQ ID NO: 44 - DNA, 11 bp, Homo sapiens
  source 1..11: mol_type = genomic DNA
agcatatatat

SEQ ID NO: 45 - DNA, 20 bp, Homo sapiens
  source 1..20: mol_type = genomic DNA
accatcccctccctatcccc

SEQ ID NO: 46 - DNA, 15 bp, Homo sapiens
  source 1..15: mol_type = genomic DNA
aaatatataatatat

SEQ ID NO: 47 - DNA, 18 bp, Homo sapiens
  source 1..18: mol_type = genomic DNA
aaggccccaccctcaggg

SEQ ID NO: 48 - DNA, 19 bp, Homo sapiens
  source 1..19: mol_type = genomic DNA
acagggccccccagctcag

SEQ ID NO: 49 - DNA, 20 bp, Homo sapiens
  source 1..20: mol_type = genomic DNA
aatgcaatggaatggaatgg

SEQ ID NO: 50 - DNA, 20 bp, Homo sapiens
  source 1..20: mol_type = genomic DNA
aacagaaccccacggcagtg

SEQ ID NO: 51 - DNA, 20 bp, Homo sapiens
  source 1..20: mol_type = genomic DNA
aatgatgcctgcatgtaggg

SEQ ID NO: 52 - DNA, 18 bp, Homo sapiens
  source 1..18: mol_type = genomic DNA
acacacacacatatatat

SEQ ID NO: 53 - DNA, 16 bp, Homo sapiens
  source 1..16: mol_type = genomic DNA
atatatatatatatcc

SEQ ID NO: 54 - DNA, 13 bp, Homo sapiens
  source 1..13: mol_type = genomic DNA
aatatatatatgg

SEQ ID NO: 55 - DNA, 14 bp, Homo sapiens
  source 1..14: mol_type = genomic DNA
aacatataatatat

SEQ ID NO: 56 - DNA, 14 bp, Homo sapiens
  source 1..14: mol_type = genomic DNA
atatatatatatcc

SEQ ID NO: 57 - DNA, 14 bp, Homo sapiens
  source 1..14: mol_type = genomic DNA
aaggagggaagggg

SEQ ID NO: 58 - DNA, 18 bp, Homo sapiens
  source 1..18: mol_type = genomic DNA
aaatatacaattatatat

SEQ ID NO: 59 - DNA, 19 bp, Homo sapiens
  source 1..19: mol_type = genomic DNA
aaacaaatactgtatttat

SEQ ID NO: 60 - DNA, 20 bp, Homo sapiens
  source 1..20: mol_type = genomic DNA
aaccaggggaggagccacag

SEQ ID NO: 61 - DNA, 18 bp, Homo sapiens
  source 1..18: mol_type = genomic DNA
acatatatacatatatgt

SEQ ID NO: 62 - DNA, 15 bp, Homo sapiens
  source 1..15: mol_type = genomic DNA
aatgtatacatatat

SEQ ID NO: 63 - DNA, 14 bp, Homo sapiens
  source 1..14: mol_type = genomic DNA
atatatatatatcc

SEQ ID NO: 64 - DNA, 16 bp, Homo sapiens
  source 1..16: mol_type = genomic DNA
atatatatgatatatc

SEQ ID NO: 65 - DNA, 18 bp, Homo sapiens
  source 1..18: mol_type = genomic DNA
aatacatatataatatat

SEQ ID NO: 66 - DNA, 20 bp, Homo sapiens
  source 1..20: mol_type = genomic DNA
aacaaccccgaacagacagt

SEQ ID NO: 67 - DNA, 18 bp, Homo sapiens
  source 1..18: mol_type = genomic DNA
acaggtctcctgggtggc

SEQ ID NO: 68 - DNA, 20 bp, Homo sapiens
  source 1..20: mol_type = genomic DNA
acaccctcctccccctgctc

SEQ ID NO: 69 - DNA, 20 bp, Homo sapiens
  source 1..20: mol_type = genomic DNA
accatcacctccccacctcc

SEQ ID NO: 70 - DNA, 20 bp, Homo sapiens
  source 1..20: mol_type = genomic DNA
acacatatacatatatatat

SEQ ID NO: 71 - DNA, 14 bp, Homo sapiens
  source 1..14: mol_type = genomic DNA
acacagcctccctc

SEQ ID NO: 72 - DNA, 17 bp, Homo sapiens
  source 1..17: mol_type = genomic DNA
accccagagatgcagag

SEQ ID NO: 73 - DNA, 20 bp, Homo sapiens
  source 1..20: mol_type = genomic DNA
aaatggactataaccacgcc

SEQ ID NO: 74 - DNA, 12 bp, Homo sapiens
  source 1..12: mol_type = genomic DNA
aaatatatatat

SEQ ID NO: 75 - DNA, 18 bp, Homo sapiens
  source 1..18: mol_type = genomic DNA
acatatatacatatatat

SEQ ID NO: 76 - DNA, 16 bp, Homo sapiens
  source 1..16: mol_type = genomic DNA
acacatacatatatat

SEQ ID NO: 77 - DNA, 20 bp, Homo sapiens
  source 1..20: mol_type = genomic DNA
acactgaccatccatagtcc

SEQ ID NO: 78 - DNA, 18 bp, Homo sapiens
  source 1..18: mol_type = genomic DNA
accaccaccaccaccatc

SEQ ID NO: 79 - DNA, 20 bp, Homo sapiens
  source 1..20: mol_type = genomic DNA
aaggaaggaaggaagggagg

SEQ ID NO: 80 - DNA, 10 bp, Homo sapiens
  source 1..10: mol_type = genomic DNA
aaagagagag

SEQ ID NO: 81 - DNA, 18 bp, Homo sapiens
  source 1..18: mol_type = genomic DNA
acagtgaggaccatgggg

SEQ ID NO: 82 - DNA, 13 bp, Homo sapiens
  source 1..13: mol_type = genomic DNA
aaatgggaagggg

SEQ ID NO: 83 - DNA, 16 bp, Homo sapiens
  source 1..16: mol_type = genomic DNA
aaggaagggaaggagg

SEQ ID NO: 84 - DNA, 12 bp, Homo sapiens
  source 1..12: mol_type = genomic DNA
aaaaatatatat

SEQ ID NO: 85 - DNA, 18 bp, Homo sapiens
  source 1..18: mol_type = genomic DNA
aaatatatatacatatat

SEQ ID NO: 86 - DNA, 19 bp, Homo sapiens
  source 1..19: mol_type = genomic DNA
aatatagactatattatat

SEQ ID NO: 87 - DNA, 19 bp, Homo sapiens
  source 1..19: mol_type = genomic DNA
aatatagactatattatat

SEQ ID NO: 88 - DNA, 14 bp, Homo sapiens
  source 1..14: mol_type = genomic DNA
acatatacggatat

SEQ ID NO: 89 - DNA, 12 bp, Synthetic construct
  source 1..12: mol_type = other DNA
gaaagaaagaaa

SEQ ID NO: 90 - DNA, 12 bp, Synthetic construct
  source 1..12: mol_type = other DNA
ggaaggaaggaa

(19) United States
(12) Patent Application Publication
(10) Pub. No.: US 2023/0322853 A1
(43) Pub. Date: Oct. 12, 2023

(54) COMPOUNDS AND COMPOSITIONS FOR THE TREATMENT OF PROLIFERATIVE DISEASES AND DISORDERS

(71) Applicant: The Board of Trustees of the Leland Stanford Junior University, Stanford, CA (US)

(72) Inventors: Graham Erwin, Stanford, CA (US); Michael P. Snyder, Stanford, CA (US)

(21) Appl. No.: 18/297,353

(22) Filed: Apr. 7, 2023

Related U.S. Application Data
(60) Provisional application No. 63/329,100, filed on Apr. 8, 2022.

(51) Int. Cl. C07K 5/117; A61P 35/00; A61P 13/08; A61P 13/12; A61P 1/16; A61P 15/00

(52) U.S. Cl. CPC C07K 5/1024; A61P 35/00; A61P 13/08; A61P 13/12; A61P 1/16; A61P 15/00; A61K 38/00

(57) ABSTRACT

Disclosed herein are compounds (e.g., cancer) comprising a moiety that specifically binds to oligonucleotides comprising one or more repeats of GAAA (e.g., a pyrrole/imidazole polyamide) and bromodomain inhibitor and compositions and methods thereof for use in treating proliferative diseases and disorders.

### CROSS-REFERENCE TO RELATED APPLICATIONS

This application claims the benefit of U.S. Provisional Application No. 63/329,100, filed Apr. 8, 2022, the content of which is herein incorporated by reference in its entirety.

### SEQUENCE LISTING STATEMENT

The contents of the electronic sequence listing titled 40727_202_SequenceListing.xml (Size: 79,563 bytes; and Date of Creation: Mar. 24, 2023) is herein incorporated by reference in its entirety.

### STATEMENT REGARDING FEDERALLY SPONSORED RESEARCH

This invention was made with Government support under contracts HG011467, HG007735, and CA233311 awarded by the National Institutes of Health. The Government has certain rights in the invention.

### TECHNICAL FIELD

Disclosed herein are compounds, compositions comprising the compounds, and uses of the compounds for treating proliferative diseases and disorders (e.g., cancer) comprising a moiety that specifically binds to oligonucleotides comprising one or more repeats of GAAA (e.g., a pyrrole/imidazole polyamide) and a bromodomain inhibitor.

### BACKGROUND

Cancer remains one of the deadliest threats to human health. Cancers, or malignant tumors, metastasize and grow rapidly in an uncontrolled manner, making timely detection and treatment extremely difficult. In the U.S., cancer affects nearly 1.9 million new patients each year and is the second leading cause of death next to heart disease. For example, kidney cancer is among the top ten most common cancers in both men and women, and the rate of new kidney cancers has been rising since the 1990s for unknown reasons. Renal cell carcinoma, in particular, is the ninth most commonly occurring cancer type in men in the US. Despite the significant advancement in the treatment of cancer, improved therapies and diagnostic methods are still being sought.

### SUMMARY

In one aspect, disclosed herein is a compound, or a pharmaceutically acceptable salt thereof, having the structure:

A-B-C
wherein:
A is a bromodomain inhibitor;
B is a linker; and
C is a moiety that specifically binds to oligonucleotides comprising one or more repeats of GAAA.

In some embodiments, C comprises a polyamide that specifically binds to oligonucleotides comprising one or more repeats of GAAA.

In some embodiments, C is

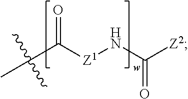

w is 5, 6, 7, 8, 9, or 10; each Z¹ is independently selected from

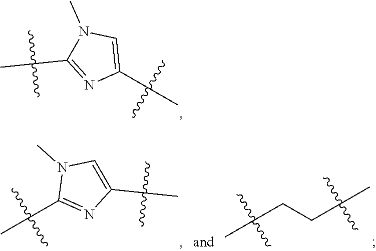

### and Z² is

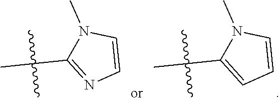

In some embodiments, C is

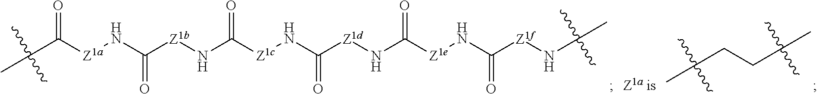

Z^{1b} and Z^{1f}

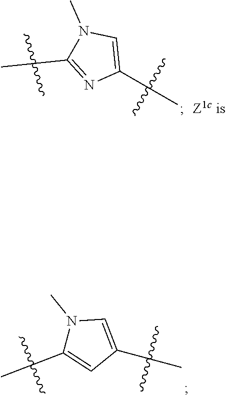

are and Z^{1d} and Z^{1e} are each independently selected from

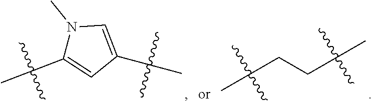

In some embodiments, C further comprises one or more

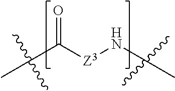

groups wherein Z³ is selected from

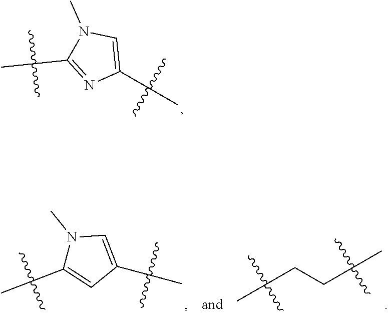

In some embodiments, C is selected from the group consisting of:

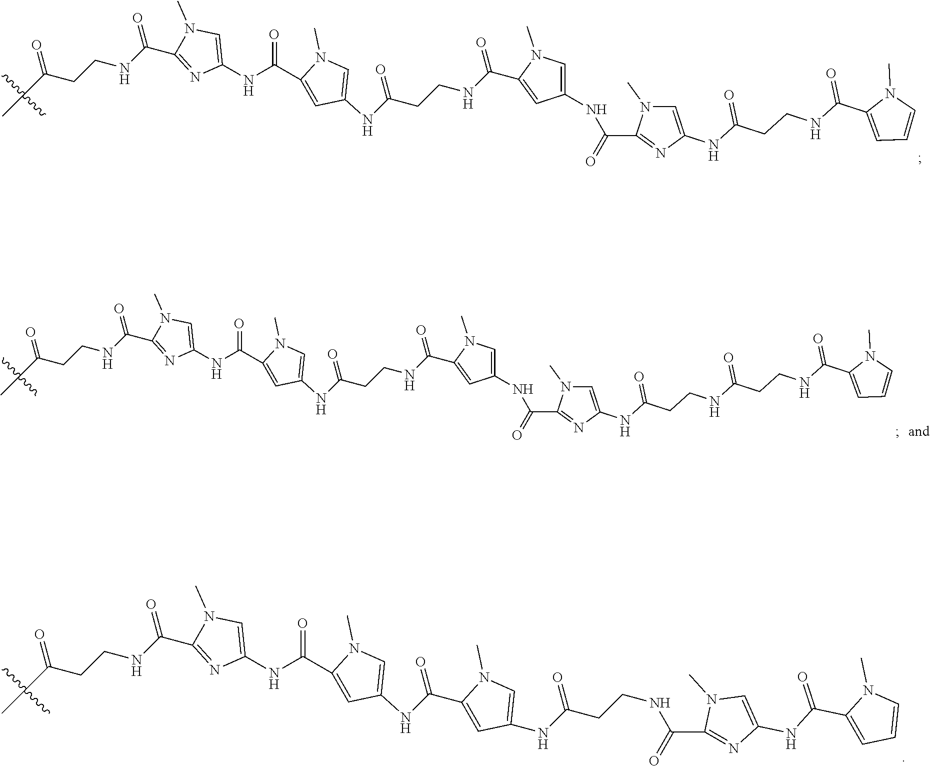 [SMILES: *C(=O)CCNC(=O)c1nc(NC(=O)c2cc(NC(=O)CCNC(=O)c3cc(NC(=O)c4nc(NC(=O)CCNC(=O)c5cccn5C)cn4C)cn3C)cn2C)cn1C ; *C(=O)CCNC(=O)c1nc(NC(=O)c2cc(NC(=O)CCNC(=O)c3cc(NC(=O)c4nc(NC(=O)CCNC(=O)CCNC(=O)c5cccn5C)cn4C)cn3C)cn2C)cn1C ; *C(=O)CCNC(=O)c1nc(NC(=O)c2cc(NC(=O)c3cc(NC(=O)CCNC(=O)c4nc(NC(=O)c5cccn5C)cn4C)cn3C)cn2C)cn1C]

In some embodiments, the bromodomain inhibitor is a bromodomain and extraterminal motif (BET) inhibitor.

In some embodiments, A is a group of formula (i):

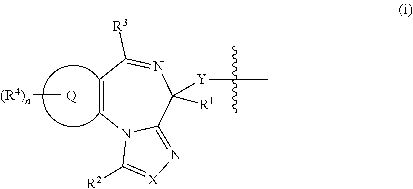

wherein:
Q is a monocyclic 5- or 6-membered heteroaryl having 1, 2, 3, or 4 heteroatoms independently selected from N, O, and S, or phenyl;
R¹ is hydrogen, halogen, or C₁-C₆ alkyl;
R² is hydrogen, C₁-C₆ alkyl, hydroxy-C₁-C₆-alkyl, amino-C₁-C₆-alkyl, C₁-C₆ alkoxy-C₁-C₆-alkyl, halo-C₁-C₆-alkyl, hydroxy, C₁-C₆-alkoxy, or —COO—R³;

R³ is hydrogen, C₁-C₆ alkyl, C₄-C₆ cycloalkyl, C₄-C₆ heterocyclyl, C₄-C₁₀ aryl, or C₄-C₁₀ heteroaryl, wherein each alkyl, cycloalkyl, heterocyclyl, aryl, or heteroaryl is optionally substituted with 1, 2, 3, 4, or 5 substituents independently selected from halo, C₁-C₆ alkyl, C₄-C₆ cycloalkyl, and C₁-C₄ haloalkyl;

n is 1, 2, or 3;

each R⁴ is independently selected from hydrogen, C₁-C₆ alkyl, halo-C₁-C₆-alkyl, C₄-C₆ cycloalkyl, C₄-C₆ heterocyclyl, C₄-C₁₀ aryl, and C₄-C₁₀ heteroaryl, wherein each cycloalkyl, heterocyclyl, aryl, or heteroaryl is optionally substituted; or any two R⁴ are taken together with the atoms to which they are attached to form an optionally substituted 5- or 6-membered ring;

X is N or CR⁵;

R⁵ is hydrogen, C₁-C₆ alkyl, C₄-C₆ cycloalkyl, C₄-C₆ heterocyclyl, C₄-C₁₀ aryl, or C₄-C₁₀ heteroaryl;

Y is —C₁-C₆ alkylene-Z— wherein Z is a bond, —C(O)O—, —C(O)—, —S(O)₂—, or —NR⁶—; and R⁶ is hydrogen or C₁-C₆ alkyl.

In some embodiments, A is a group of formula (ia):

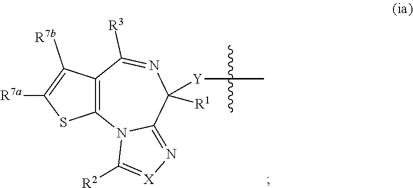

and R^{7a} and R^{7b} are independently selected from hydrogen, C₁-C₆ alkyl, halo-C₁-C₆-alkyl, C₄-C₆ cycloalkyl, C₄-C₆ heterocyclyl, C₄-C₁₀ aryl, or C₄-C₁₀ heteroaryl.

In some embodiments, X is N. In some embodiments, Y is —C₁-C₆ alkylene-C(O)—. In some embodiments, Y is —CH₂—C(O)—. In some embodiments, R¹ is hydrogen, methyl, ethyl, or n-propyl. In some embodiments, R¹ is hydrogen. In some embodiments, R² is hydrogen or C₁-C₆ alkyl. In some embodiments, R² is C₁-C₆ alkyl. In some embodiments, R² is methyl. In some embodiments, R³ is phenyl, optionally substituted with 1, 2, 3, 4, or 5 substituents independently selected from halo, C₁-C₆ alkyl, C₄-C₆ cycloalkyl, C₁-C₄ haloalkyl. In some embodiments, R³ is 4-chlorophenyl. In some embodiments, R^{7a} and R^{7b} are each selected from hydrogen, C₁-C₆ alkyl, and halo-C₁-C₆-alkyl. In some embodiments, both of R^{7a} and R^{7b} are C₁-C₆ alkyl. In some embodiments, both of R^{7a} and R^{7b} are methyl.

In some embodiments, the group of formula (i) is:

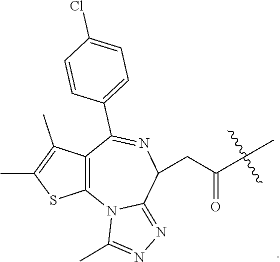 [SMILES: *C(=O)CC1N=C(c2ccc(Cl)cc2)c2c(sc(C)c2C)-n2c(C)nnc21]

In some embodiments, B comprises one or more groups selected from —C(R′)₂—, —CH═CH—, —C≡C—, —O—, —NR′—, —BR′—, —S—, —C(O)—, —C(NR′)—, —S(O)—, —S(O)₂—, arylene, heteroarylene, cycloalkylene, and heterocyclylene, wherein each R′ is independently selected from hydrogen, C₁-C₆ alkyl, C₂-C₆ alkenyl, C₂-C₆ alkynyl, aryl, arylalkyl, cycloalkyl, cycloalkylalkyl, heterocyclyl, heterocyclyl, heteroaryl, and heteroarylalkyl, and wherein each alkyl, alkenyl, alkynyl, arylene, heteroarylene, cycloalkylene, and heterocyclylene is independently unsubstituted or substituted with 1, 2, or 3 substituents. In some embodiments, B comprises a combination of one or more groups selected from —O—, —CH₂—, —C(O)—, and —NR′—.

In some embodiments, B is —NR′—(CH₂CH₂O)ₘ—(CH₂)ₚ—C(O)NR′—(CH₂)_{q}—NR′—(CH₂)ᵣ—NR′—; m, p, q, and r are each independently selected from 1, 2, 3, 4, 5, 6, 7, 8, 9, or 10; and each R′ is selected from hydrogen or C₁-C₆ alkyl.

In some embodiments, B is —NH—(CH₂CH₂O)₆—(CH₂)₂—C(O)NH—(CH₂)₃—N(CH₃)—(CH₂)₃—NH—.

In some embodiments, the compound is selected from:

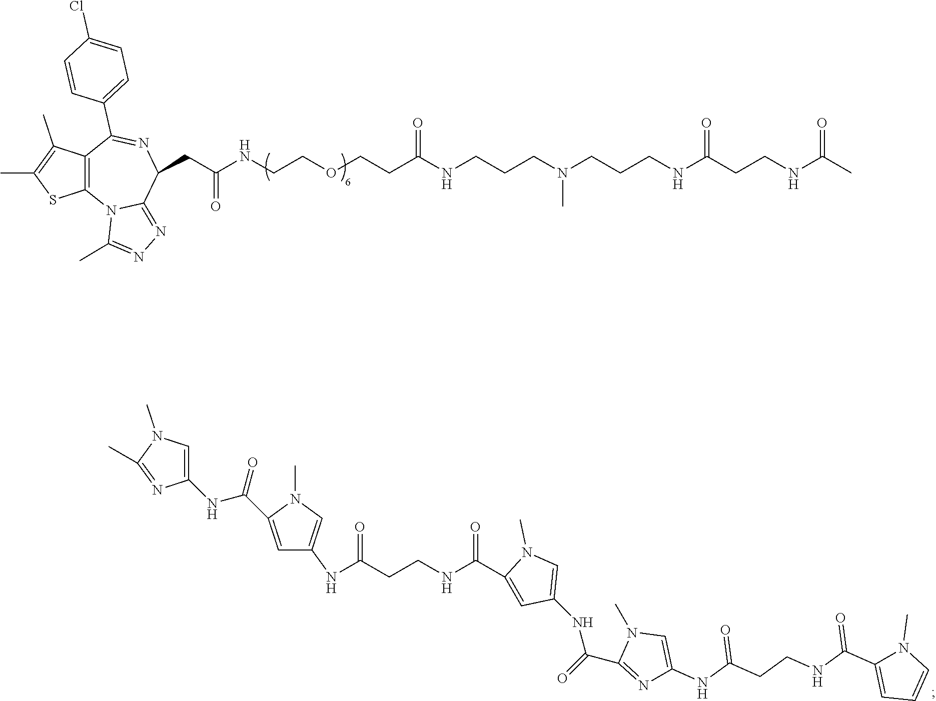

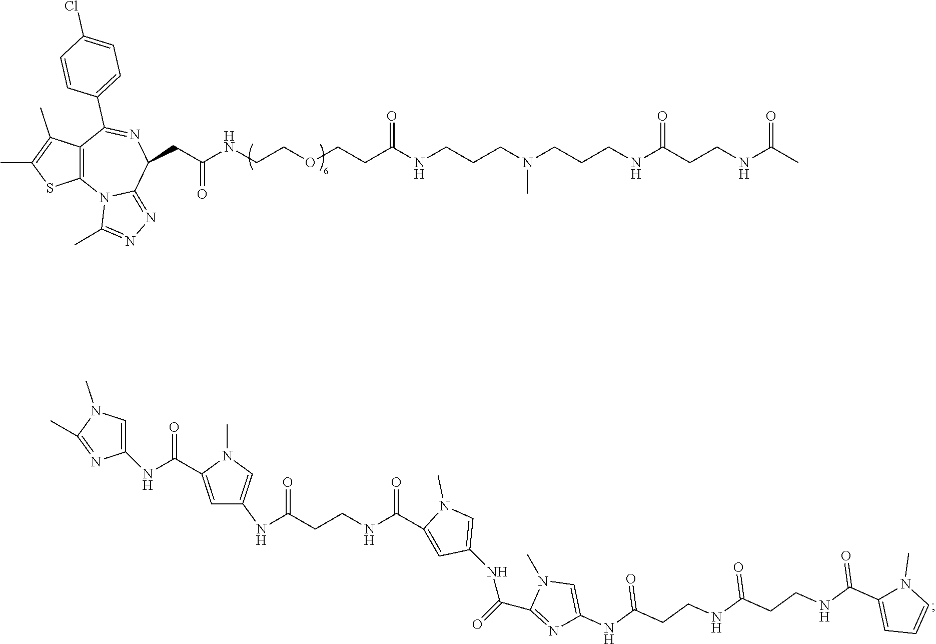

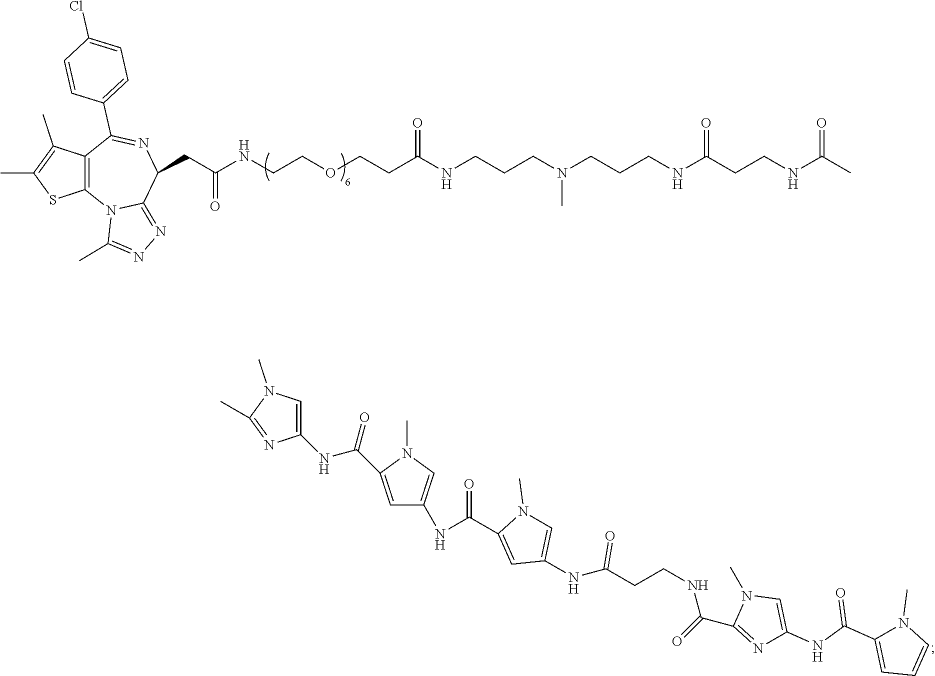

and pharmaceutically acceptable salts thereof.

In another aspect, disclosed herein is a pharmaceutical composition comprising an effective amount of a compound disclosed herein (e.g., a compound having the structure A-B-C as disclosed herein), or a pharmaceutically acceptable salt thereof, and a pharmaceutically acceptable carrier.

In another aspect, disclosed herein is a method of treating a disease or disorder in a subject in need thereof, comprising administering to the subject an effective amount of a compound disclosed herein (e.g., a compound having the structure A-B-C as disclosed herein), or a pharmaceutically acceptable salt thereof, or a pharmaceutical composition comprising a compound disclosed herein (e.g., a compound having the structure A-B-C as disclosed herein), or a pharmaceutically acceptable salt thereof.

In some embodiments, the disease or disorder is characterized by a disease related gene comprising a nucleic acid sequence having an expansion of GAAA repeats. In some embodiments, the nucleic acid sequence of the disease related gene comprises at least 50 GAAA repeats. In some embodiments, expression of the gene comprising a nucleic acid sequence having an expansion of GAAA repeats is increased compared to prior to administration. In some embodiments, the disorder is a proliferative disease. In some embodiments, the disease or disorder comprises a cancer. In some embodiments, the cancer comprises kidney cancer, liver cancer, prostate cancer, or ovarian cancer. In some embodiments, the cancer is cancer of the kidney. In some embodiments, the cancer is a carcinoma.

Other aspects and embodiments of the disclosure will be apparent in light of the following detailed description and accompanying figures.

### BRIEF DESCRIPTION OF THE DRAWINGS

FIGS. 1A-1E show genome-wide detection of recurrent repeat expansions (rREs) in cancer genomes. FIG. 1A is a scheme of method to identify rREs in 2509 patients across 29 human cancers. 1, squamous cell carcinoma (Head-SCC); 2, Skin-Melanoma; 3, glioblastoma (CNS-GBM); 4 medulloblastoma (CNS-Medullo); 5, pilocytic astrocytoma (CNS-PiloAstro); 6, esophageal adenocarcinoma (Eso-AdenoCA), 7, osteosarcoma (Bone-Osteosarc); 8, leiomyosarcoma (Bone-Leiomyo); 9, thyroid adenocarcinoma (Thy-AdenoCA); 10, Lung-AdenoCA; 11, Lung-SCC; 12, Breast-AdenoCA; 13, B-cell non-Hodgkin lymphoma (Lymph-BNHL); 14, chronic lymphocytic leukemia (Lymph-CLL); 15, acute myeloid leukemia (Myeloid-AML); 16, myeloproliferative neoplasm (Myeloid-MPN); 17, Biliary-AdenoCA; 18, hepatocellular carcinoma (Liver-HCC); 19, Stomach-AdenoCA; 20, pancreatic (Panc-AdenoCA), 21, Panc-Endocrine; 22, colorectal (ColoRect-AdenoCA); 23, prostate (Prost-AdenoCA); 24, chromophobe renal cell carcinoma (Kidney-ChRCC); 25, renal cell carcinoma (Kidney-RCC); 26, papillary renal cell carcinoma (Kidney-pRCC); 27, Uterus-AdenoCA; 28, Ovary-AdenoCA; 29, Bladder-TCC. FIG. 1B is a graph of the distribution of rREs across cancer types. FIG. 1C shows the proportion of cancer genomes with rREs. FIG. 1D is a graph of STR mutation rate for cancer genomes with and without a rRE. Two-tailed Wilcoxon rank sum test. FIG. 1E is a graph of distribution of rREs across microsatellite stable (MSS) and microsatellite instability high (MSI-high) cancers. Chi-square test with Yates' correction.

FIGS. 2A-2F show features of rREs. FIG. 2A is a circos plot depicting (from outside to inside) p-value of rREs, location of rREs where darker shading indicates the rRE observed across 3 cancers, early and late replicating regions (yellow and purple, respectively), and simple sequence repeats. FIG. 2B is a graph of the distribution of the repeat unit (motif) for rREs. FIG. 2C is a graph of the distance of rREs to the end of the chromosome arm. FIG. 2D is a graph of the proportion of genic features that overlap with rREs. UTR, untranslated region. FIG. 2E is a graph of the distance of simple repeats and rREs to the nearest ENCODE candidate cis-regulatory element (cCRE). Welch's t-test. FIG. 2F is a graph of motifs enriched in the catalogue of rREs.

FIGS. 3A-3D show the association of rREs with cancer features. FIG. 3A shows the frequency of rREs in genes of interest, including the nine COSMIC genes. FIG. 3B shows the association of rREs with human diseases. FIG. 3C is a graph of the distance of simple repeats, non-prostate cancer rREs, and prostate-cancer rREs to the nearest prostate cancer risk locus. Statistical significance was measured with Welch's t-test. FIG. 3D is a graph of the association between SNVs in genes in the COSMIC tier 1 genes and the presence of rREs. Two-tailed Student's t test with FDR correction by the Benjamini-Hochberg method.

FIGS. 4A-4E show an rRE in Renal Cell Carcinoma (RCC). FIG. 4A is gel electrophoresis of the GAAA tandem repeat in RCC cell lines. FIG. 4B is gel electrophoresis of the GAAA tandem repeat in primary RCC samples from patients and matching normal tissue. FIG. 4C shows the locus surrounding the rRE detected in the intron of UGT2B7. Signal traces of Po12, H3K27ac, H3K4me1, and p300 in HepG2 cells are shown. Candidate cis-regulatory elements (cCREs) and chromatin states (ChromHMM) are also depicted. FIG. 4D shows the expression of UGT2B7 isoform ENST00000508661.1 in RCC samples as a function of the detection of the rRE in UGT2B7 (Normalized Expression, Counts). Significance was measured with Wald test with FDR correction (Benjamini-Hochberg). FIG. 4E is a visualization of long-read sequencing of the GAAA rRE in the intron of UGT2B7. Data are from PacBio HiFi sequencing.

FIGS. 5A-5D show the design and characterization of GAAA-targeting molecules in RCC. FIG. 5A is the chemical structures of Syn-TEF3, PA3, Syn-TEF4 and PA4. Syn-TEF3 and PA3 target 5′-AAGAAAGAA-3′ (sequence as shown SEQ ID NO: 89). Syn-TEF4 and PA4 target 5′-AAGGAAGG-3′(sequence as shown SEQ ID NO: 90). The structures of N-methylpyrrole (open circles), N-methylimidazole (filled circles), and β-alanine (diamonds) are shown. N-methylimidazole is bolded for clarity. The structure of JQ1 linked to polyethylene glycol (PEG₆) is represented as a blue circle. The structure of isophthalic acid and linker is represented as IPA. Complete chemical structures are depicted in FIG. 6. The asterisk indicates the site where the R group attaches to the polyamide. Mismatches formed with Syn-TEF4 and PA4 are indicated with orange. FIG. 5B are graphs of the relative cell density of RCC cell lines Caki-1 and 786-o following treatment (72 h) with compounds, as indicated. Relative cell density was measured with CCK-8 assay. Results are mean±SEM (n=4). Error bars omitted if smaller than the symbol. FIG. 5C is the quantification of the percentage of propidium iodide-positive cells. P values are from one-way ANOVA with Bonferroni's correction for multiple comparisons. Results are shown as the mean±s.e.m. (n=3 biological replicates except n=2 biological replicates for Syn-TEF3 in 786-O cells). FIG. 5D is images of live-cell microscopy of Caki-1 and 786-O cells stained with propidium iodide (red) and Hoechst 33342 (blue). Scale bars, 100 μm.

FIGS. 6A-6D are the chemical structures, formulas, and molecular weights of Syn-TEF3(FIG. 6A), Syn-TEF4 (FIG. 6B), PA3 (FIG. 6C), and PA4 (FIG. 6D).

FIGS. 7A-7C show Syn-TEF treatment of RCC cell lines. FIG. 7A is the quantitation of the percentage of propidium iodide-positive cells. P values are from a one-way ANOVA adjusted with Bonferroni correction for multiple comparisons. Results are mean±s.e.m. (n=3 biological replicates, except n=2 biological replicates for Syn-TEF3 in 786-O). FIG. 7B are images of live cell microscopy of Caki-1 and 786-O cells stained with propidium iodide (red) and Hoechst 33342 (blue). Scale bars, 100 μm. FIG. 7C are graphs of the relative cell density of RCC cell lines following treatment (72 h) with compounds (50 μM Syn-TEF or 0.1% DMSO vehicle, as indicated). Results are mean±s.e.m. (ACHN and RCC-4 are n=4 biological replicates, A498 and Caki-2 are n=3 biological replicates).

### DETAILED DESCRIPTION

Disclosed herein are bifunctional compounds that specifically target GAAA repeats in DNA. The compounds comprise a pyrrole/imidazole polyamide moiety and a bromodomain inhibitor, or functional fragment or variant thereof. The bromodomain inhibitor may comprise a bromodomain and extraterminal motif (BET) inhibitor (e.g., a thienotriazolodiazepine). Compounds of the disclosure can be used to treat proliferative diseases and disorders, including cancer. Pharmaceutical compositions comprising the disclosed compounds, methods of using the disclosed compounds, and kits comprising the compounds are also provided herein.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to ±10% of the indicated number. For example, “about 10%” may indicate a range of 9% to 11%, and “about 1” may mean from 0.9-1.1. Other meanings of “about” may be apparent from the context, such as rounding off; for example, “about 1” may also mean from 0.5 to 1.4.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987.

As used herein, the term “alkyl” refers to a radical of a straight or branched saturated hydrocarbon chain. The alkyl chain can include, e.g., from 1 to 24 carbon atoms (C₁-C₂₄ alkyl), 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 1 to 3 carbon atoms (C₁-C₃ alkyl), or 1 to 2 carbon atoms (C₁-C₂ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy, and tert-butoxy.

The term “alkoxyalkyl,” as used herein, refers to an alkyl group, as defined herein, in which at least one hydrogen atom (e.g., one hydrogen atom) is replaced with an alkoxy group, as defined herein. Representative examples of alkoxyalkyl include, but are not limited to, methoxymethyl.

The term “amino,” as used herein, refers to an —NH₂ group. The term “alkylamino,” as used herein, refers to a group —NHR, wherein R is an alkyl group as defined herein. The term “dialkylamino,” as used herein, refers to a group —NR₂, wherein each R is independently an alkyl group as defined herein.

The term “aminoalkyl,” as used herein, refers to an alkyl group, as defined herein, in which at least one hydrogen atom (e.g., one hydrogen atom) is replaced with an amino group.

As used herein, the term “aryl” refers to a radical of a monocyclic, bicyclic, or tricyclic 4n+2 aromatic ring system (e.g., having 6, 10, or 14 π electrons shared in a cyclic array) having 6-14 ring carbon atoms and zero heteroatoms (“C₆-C₁₄ aryl”). In some embodiments, an aryl group has six ring carbon atoms (“C₆ aryl,” e.g., phenyl). In some embodiments, an aryl group has ten ring carbon atoms (“C₁₀ aryl,” e.g., naphthyl such as 1-naphthyl and 2-naphthyl).

As used herein, the term “arylene” refers to a divalent aryl radical.

As used herein, the term “cycloalkyl” refers to a radical of a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

As used herein, the term “cycloalkylene” refers to a divalent cycloalkyl radical.

As used herein, the term “halogen” or “halo” refers to F, Cl, Br, or I.

As used herein, the term “haloalkyl” refers to an alkyl group, as defined herein, in which at least one hydrogen atom (e.g., one, two, three, four, five, six, seven or eight hydrogen atoms) is replaced with a halogen. In some embodiments, each hydrogen atom of the alkyl group is replaced with a halogen (“perhaloalkyl”). Representative examples of haloalkyl include, but are not limited to, fluoromethyl, difluoromethyl, trifluoromethyl, 2-fluoroethyl, 2,2,2-trifluoroethyl, and 3,3,3-trifluoropropyl.

As used herein, the term “heteroalkyl” refers to an alkyl group, as defined herein, in which one or more of the carbon atoms (and any associated hydrogen atoms) are each independently replaced with a heteroatom group such as —NH—, —O—, —S—, —S(O)—, —S(O)₂—, —OP(O)(O⁻)O—, or the like. By way of example, 1, 2, 3, 4, 5, 6, or more carbon atoms may be independently replaced with the same or different heteroatom group. A heteroalkyl group can also include one or more carbonyl moieties (e.g., wherein a carbon atom of the alkyl group is oxidized to a —C(O)— group).

As used herein, the term “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms, wherein each heteroatom is independently selected from nitrogen, oxygen, and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

As used herein, the term “heterocyclyl” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Exemplary 3-membered heterocyclyl groups containing one heteroatom include, without limitation, azirdinyl, oxiranyl, and thiorenyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione.

Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin-2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

As used herein, the term “heterocyclylene” refers to a divalent heterocyclyl radical.

As used herein, the term “hydroxy” or “hydroxyl” refers to an —OH group.

The term “hydroxyalkyl,” as used herein, refers to an alkyl group, as defined herein, in which at least one hydrogen atom (e.g., one hydrogen atom) is replaced with a hydroxy group.

As used herein, the term “substituent” refers to a group substituted on an atom of the indicated group.

When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, thiol, thione, or combinations thereof

As used herein, in chemical structures the indication:

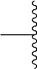

represents a point of attachment of one moiety to another moiety (e.g., a substituent group to the rest of the compound).

In some instances, the number of carbon atoms in a hydrocarbyl substituent (e.g., alkyl alkenyl) is indicated by the prefix “Cₓ-C_{y}” wherein x is the minimum and y is the maximum number of carbon atoms in the substituent. Thus, for example, “C₁-C₃ alkyl” refers to an alkyl substituent containing from 1 to 3 carbon atoms.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

When substituent groups are specified by their conventional chemical formulae, written from left to right, such indication also encompass substituent groups resulting from writing the structure from right to left. For example, if a bivalent group is shown as —CH₂O—, such indication also encompasses —OCH₂—; similarly, —OC(O)NH— also encompasses —NHC(O)O—. When linker moieties are shown, the linkers can be attached to other moieties of the compound in either direction.

The terms “administer,” “administering,” or “administration,” as used herein refer to implanting, absorbing, ingesting, injecting, inhaling, or otherwise introducing a compound or a pharmaceutical composition.

As used herein, the terms “condition,” “disease,” and “disorder” are used interchangeably.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. The nucleic acid, whether DNA or RNA may comprise non-natural nucleotides, modified nucleotides, and/or non-nucleotide building blocks that can exhibit the same function as natural nucleotides (e.g., “nucleotide analogs”). Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non- nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof

An “effective amount” of a compound or composition refers to an amount sufficient to elicit a desired biological response (e.g., treating a condition). As will be appreciated by those skilled in the art, the effective amount of a compound may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of a compound or composition may reduce tumor burden or stop the growth or spread of a tumor.

A “therapeutically effective amount” of a compound or composition is an amount sufficient to provide a therapeutic benefit in the treatment of a condition, or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “subject” to which administration is contemplated includes, but is not limited to, a human (e.g., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys).

As used herein, the terms “treatment,” “treat,” and “treating” refer to reversing, alleviating, delaying the onset of, or inhibiting the progress of a disease or condition, or one or more signs or symptoms thereof In some embodiments, “treatment,” “treat,” and “treating” require that signs or symptoms of the disease disorder or condition have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

### Compounds

In one aspect disclosed herein is a compound having the structure:

A-B-C
wherein:
A is a bromodomain inhibitor;
B is a linker; and
C is a moiety that specifically binds to oligonucleotides comprising one or more repeats of GAAA.

The compound includes C, which is a moiety that specifically binds to oligonucleotides comprising one or more repeats of GAAA. The moiety that specifically binds to oligonucleotides comprising one or more repeats of GAAA may be any molecule, compound, or fragment thereof which specifically binds to a nucleic acid sequence of (GAAA), where n is an integer from 1 to 50 or any range or integer therebetween (e.g., 1, 2, 3, 4, 5, 6, 10, 15, 20, 25, 30, 35, 40, or 45).

“Specifically binds” or “specific binding” when referring to a moiety that specifically binds to oligonucleotides comprising one or more repeats of GAAA means a moiety that specifically binds sequences comprising (GAAA)ₙ with greater affinity than for other nucleic acid sequences or repeat expansions. Typically, the moiety binds to a (GAAA)ₙ repeat sequence with a dissociation constant (K_{D}) of about 1×10⁻⁸ M or less, for example about 1×10⁻⁹ M or less, about 1×10⁻¹⁰ M or less, about 1×10⁻¹¹ M or less, or about 1×10⁻¹² M or less. Generally, the K_{D} is at least one hundred fold less than its K_{D} for binding to another nucleic acid sequence. Thus, under designated conditions (e.g., cellular or in vivo conditions) the moiety binds to its particular “target” sequence and does not bind in any significant amount to other nucleic acids present. As such, the moiety disclosed herein would not bind in any significant amount to other sequences comprising repeats of GAA or CAAA due to the much lower dissociation constant, which is a consequence of the sequence and length specificity of the moiety. For example, one four nucleotide repeat created by GAA is GAAG, which would not be bound by a moiety having specificity for GAAA.

C may comprise a nucleic acid, such as a DNA or RNA aptamer, small peptides, polyamides, antibodies or antibody fragments, and small molecules such as small chemical compounds. In some embodiments, C comprises a polyamide that specifically binds to oligonucleotides comprising one or more repeats of GAAA.

In some embodiments, C is a group represented by the following formula:

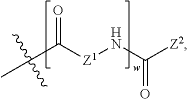

wherein w is 5, 6, 7, 8, 9, or 10; each Z¹ is independently selected from

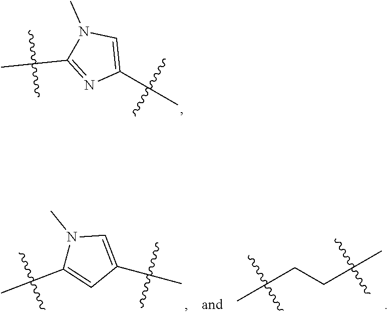

### and Z² is

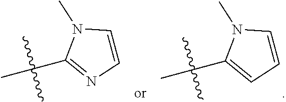

In some embodiments, C comprises

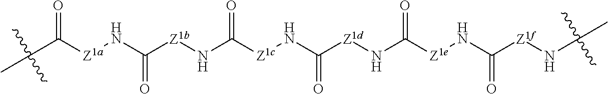

wherein Z^{1a}, Z^{1b}, Z^{1c}, Z^{1d}, Z^{1e}, and Z^{1f} are each independently selected from

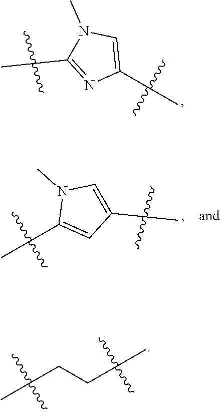

In some embodiments, Z^{1a} is

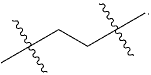 [SMILES: *CC*]

In some embodiments, Z^{1b} is

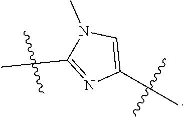 [SMILES: *c1cn(C)c(*)n1]

In some embodiments, Z^{1f} is

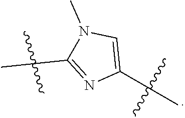 [SMILES: *c1cn(C)c(*)n1]

In some embodiments, Z^{1b} and Z^{1f} are both

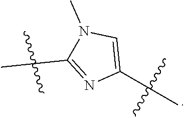 [SMILES: *c1cn(C)c(*)n1]

In some embodiment, Z^{1a} is

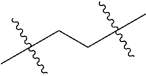 [SMILES: *CC*]

and Z^{1b} and Z^{1f} are both

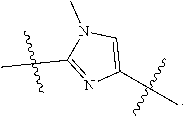 [SMILES: *c1cn(C)c(*)n1]

In some embodiments, Z^{1c} is

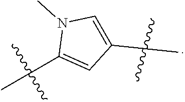 [SMILES: *c1cc(*)n(C)c1]

In some embodiments, Z^{1c} is

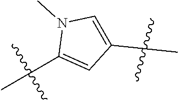 [SMILES: *c1cc(*)n(C)c1]

### and Z^{1a} is

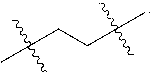 [SMILES: *CC*]

In some embodiments, Z^{1c} is

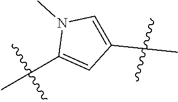 [SMILES: *c1cc(*)n(C)c1]

and one or both of Z^{1b} and Z^{1f} are

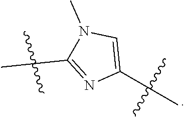 [SMILES: *c1cn(C)c(*)n1]

In some embodiments, Z^{1c} is

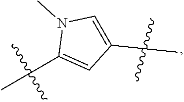 [SMILES: *c1cc(*)n(C)c1]

### Z^{1a} is

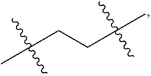 [SMILES: *CC*]

and Z^{1b} and Z^{1f} are both

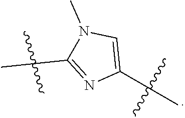 [SMILES: *c1cn(C)c(*)n1]

In some embodiments, Z^{1d} and Z^{1e} are independently selected from

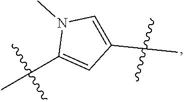 [SMILES: *c1cc(*)n(C)c1]

or

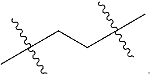 [SMILES: *CC*]

In some embodiments, Z^{1d} is

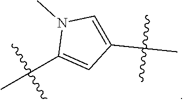 [SMILES: *c1cc(*)n(C)c1]

In some embodiments, Z^{1d} is

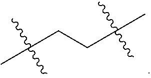 [SMILES: *CC*]

In some embodiments, Z^{1e} is

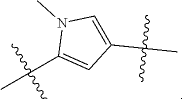 [SMILES: *c1cc(*)n(C)c1]

In some embodiments, Z^{1e} is

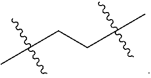 [SMILES: *CC*]

In some embodiments, both Z^{1d} and Z^{1e} are

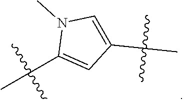 [SMILES: *c1cc(*)n(C)c1]

In some embodiments, both Z^{1d} and Z^{1e} are

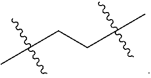 [SMILES: *CC*]

In some embodiments, C further comprises one or more

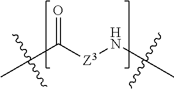

groups wherein each Z³ is independently selected from

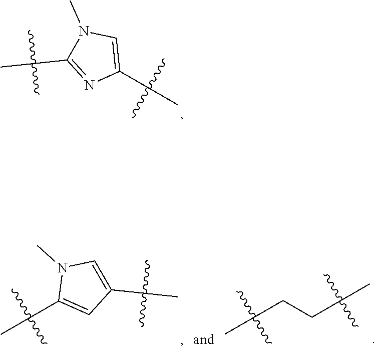

In select embodiments, C is selected from the group consisting of:

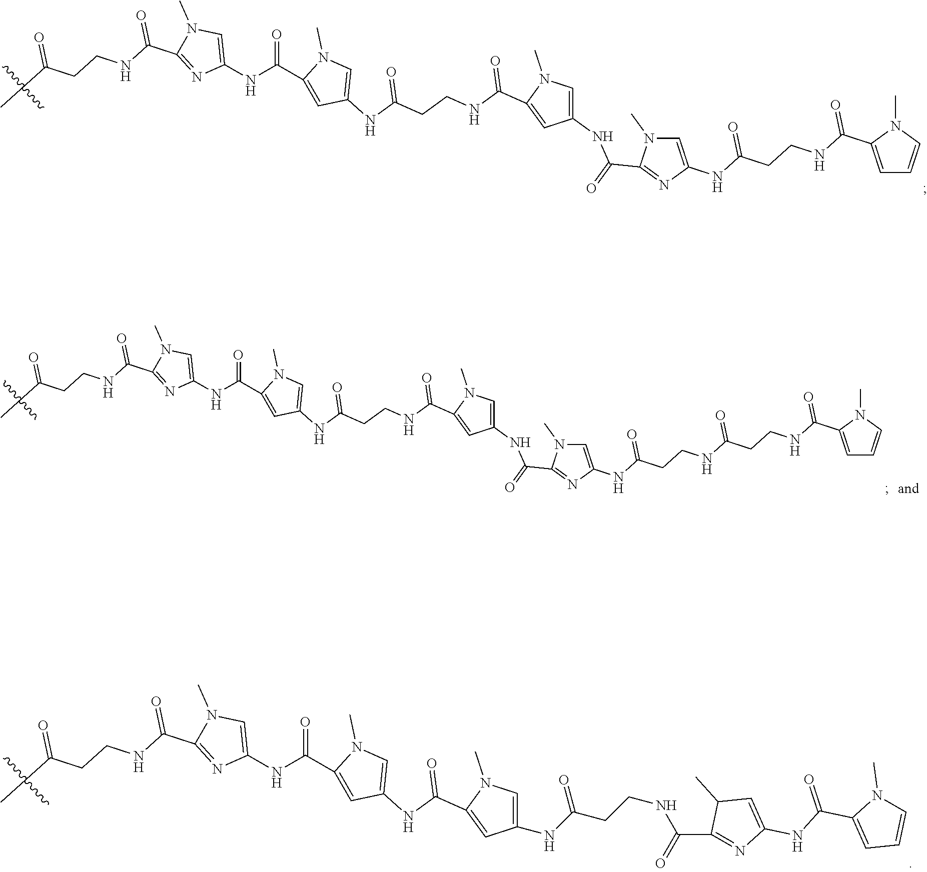 [SMILES: *C(=O)CCNC(=O)c1nc(NC(=O)c2cc(NC(=O)CCNC(=O)c3cc(NC(=O)c4nc(NC(=O)CCNC(=O)c5cccn5C)cn4C)cn3C)cn2C)cn1C ; *C(=O)CCNC(=O)c1nc(NC(=O)c2cc(NC(=O)CCNC(=O)c3cc(NC(=O)c4nc(NC(=O)CCNC(=O)CCNC(=O)c5cccn5C)cn4C)cn3C)cn2C)cn1C ; *C(=O)CCNC(=O)c1nc(NC(=O)c2cc(NC(=O)c3cc(NC(=O)CCNC(=O)C4=NC(NC(=O)c5cccn5C)=CC4C)cn3C)cn2C)cn1C]

The compound includes A, which is a bromodomain inhibitor. The bromodomain inhibitor is any molecule or compound that can prevent or inhibit, in part or in whole, the binding of at least one bromodomain to acetyl-lysine residues of proteins (e.g., to the acetyl-lysine residues of histones). The bromodomain inhibitor may be any molecule or compound that inhibits a bromodomain as described above, including nucleic acids such as DNA and RNA aptamers, antisense oligonucleotides, siRNA and shRNA, small peptides, antibodies or antibody fragments, and small molecules such as small chemical compounds. It is to be understood that the bromodomain inhibitor may inhibit only one bromo-domain-containing protein or it may inhibit more than one or all bromodomain-containing proteins.

Examples of bromodomain inhibitors are described in JP 2009028043, JP 2009183291, WO 2012/075383, WO 2011/054553, WO 2011/054846, WO 2011/054843, WO 2011/054844, WO 2011/054848, WO2011/143651, WO2009/084693A1, WO2009/084693, US 2012028912, Filippakopoulos et al. Bioorg Med Chem. 20(6): 1878-1886, 2012; Chung et al. J. Med. Chem. 54(11):3827-38, 2011; and Chung et al. J. Biomol. Screen. 16(10): 1170-85, 2011, which are incorporated herein by reference.

In some embodiments, the bromodomain inhibitor is a bromodomain and extraterminal motif (BET) inhibitor. A BET inhibitor is any molecule or compound that can prevent or inhibit the binding of the bromodomain of at least one BET family member to acetyl-lysine residues of proteins. BET family members include polypeptides comprising two bromodomains and an extraterminal (ET) domain or a fragment thereof having transcriptional regulatory activity or acetylated lysine binding activity. The BET family of human bromodomains are transcriptional co-activators involved in cell cycle progression, transcriptional activation and elongation. Exemplary BET family members include Brd2, Brd3, Brd4 and BrdT. Brd4 is a member of the BET family of bromodomain-containing proteins that bind to acetylated histones to influence transcription. The BET inhibitor may be any molecule or compound that inhibits a BET as described above, including nucleic acids such as DNA and RNA aptamers, antisense oligonucleotides, siRNA and shRNA, small peptides, antibodies or antibody fragments, and small molecules such as small chemical compounds.

Examples of BET inhibitors are described in W02009/084693, WO 2011/161031, WO 2011/143669, WO 2011/143660, WO 2011/054845, WO 2011/054851, WO 2011/054841, WO 2014/159392, WO 2015/013635, WO 2015/117083, WO 2015/117053, WO 2015/117055, WO 2015/117087, and JP 2008156311, which are incorporated herein by reference. It is to be understood that a BET inhibitor may inhibit only one BET family member or it may inhibit more than one or all BET family members. Examples of BET inhibitors known in the art include, but are not limited to, RVX-208 (Resverlogix), PFI-1 (Structural Genomics Consortium), OTX015 (Mitsubishi Tanabe Pharma Corporation), BzT- Glaxo SmithKline).

In some embodiments, the BET inhibitor is a thienotriazolodiazepine compound that inhibits BET family polypeptides by competitively binding to the acetyl-lysine recognition pocket. In some embodiments, the BET inhibitor is (S)-tert-butyl 2-(4-(4-chlorophenyl)-2,3,9-trimethyl-6H-thieno[3,2-f][1,2,4]triazolo[4,3-a][1,4]diazepin-6-yl)acetate (“JQ1”), or an analog or variant thereof.

In some embodiments, A is a group of formula (i):

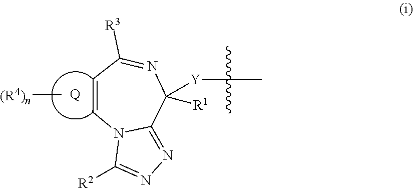

wherein
Q is a monocyclic 5- or 6-membered heteroaryl having 1, 2, 3, or 4 heteroatoms independently selected from N, O, and S, or phenyl;
R¹ is hydrogen, halogen, or C₁-C₆ alkyl;
R² is hydrogen, C₁-C₆ alkyl, hydroxy-C₁-C₆-alkyl, amino-C₁-C₆-alkyl, C₁-C₆-alkoxy-C₁-C₆-alkyl, halo-C₁-C₆-alkyl, hydroxy, C₁-C₆-alkoxy, or —COO—R³;
R³ is hydrogen, C₁-C₆ alkyl, C₄-C₆ cycloalkyl, C₄-C₆ heterocyclyl, C₄-C₁₀ aryl, or C₄-C₁₀ heteroaryl, wherein each alkyl, cycloalkyl, heterocyclyl, aryl or heteroaryl is optionally substituted with 1, 2, 3, 4, or 5 substituents independently selected from halo, C₁-C₆ alkyl, C₄-C₆ cycloalkyl, and C₁-C₄ haloalkyl;
n is 1, 2, or 3;
each R⁴ is independently selected from hydrogen, C₁-C₆ alkyl, halo-C₁-C₆-alkyl, C₄-C₆ cycloalkyl, C₄-C₆ heterocyclyl, C₄-C₁₀ aryl, and C₄-C₁₀ heteroaryl, wherein each cycloalkyl, heterocyclyl, aryl or heteroaryl is optionally substituted; or any two R⁴ are taken together with the atoms to which they are attached to form an optionally substituted 5- or 6-membered ring;
X is N or CR⁵;
R⁵ is hydrogen, C₁-C₆ alkyl, C₄-C₆ cycloalkyl, C₄-C₆ heterocyclyl, C₄-C₁₀ aryl, or C₄-C₁₀ heteroaryl;
Y is —C₁-C₆ alkylene-Z—, wherein Z is a bond, —C(O)O—, —C(O)—, —S(O)₂—, or —NR⁶—; and
R⁶ is hydrogen or C₁-C₆ alkyl.

In some embodiments, Q is a monocyclic heteroaryl having 1 heteroatom selected from N, O, and S (i.e., Q is a thiophene, furan, or pyrazole ring). In some embodiments, Q is phenyl.

In some embodiments, A is a group of formula (ia):

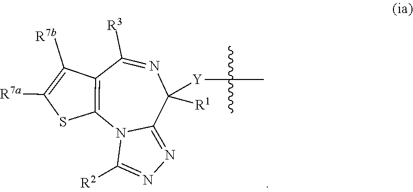

wherein
R^{7a} and R^{7b} are independently selected from C₁-C₆ alkyl, hydrogen, halo-C₁-C₆-alkyl, C₄-C₆ cycloalkyl, C₄-C₆ heterocyclyl, C₄-C₁₀ aryl, and C₄-C₁₀ heteroaryl.

In some embodiments, X is N. In some embodiments, X is CR⁵ wherein R⁵ is hydrogen, C₁-C₆ alkyl, C₄-C₆ cycloalkyl, C₄-C₆ heterocyclyl, C₄-C₁₀ aryl, or C₄-C₁₀ heteroaryl.

In some embodiments, Y is —C₁-C₆ alkylene-, —C₁-C₆ alkylene-C(O)O—, —C₁-C₆ alkylene-C(O)—, —C₁-C₆ alkylene-S(O)₂—, —C₁-C₆ alkylene-NH—, or —C₁-C₆ alkylene-N(C₁-C₆ alkyl)-. In some embodiments, Y is —C₁-C₆ alkylene-C(O)—. In some embodiments, Y is —CH₂—C(O)—.

In some embodiments, R¹ is hydrogen or C₁-C₆ alkyl. In some embodiments, R¹ is hydrogen, methyl, ethyl, or n-propyl. In some embodiments, R¹ is hydrogen. In some embodiments, R¹ is hydrogen and Y is —CH₂—C(O)—. In some embodiments, R¹ is methyl, ethyl, or n-propyl and Y is —CH₂—C(O)—.

In some embodiments, R² is hydrogen or C₁-C₆ alkyl. In some embodiments, R² is C₁-C₆ alkyl. In some embodiments, R² is methyl.

In some embodiments, R³ is C₄-C₁₀ aryl, which is optionally substituted with 1, 2, 3, 4, or 5 substituents independently selected from halo, C₁-C₆ alkyl, C₄-C₆ cycloalkyl, and C₁-C₄ haloalkyl. In some embodiments, R³ is phenyl, optionally substituted with 1, 2, 3, 4, or 5 substituents independently selected from halo, C₁-C₆ alkyl, C₄-C₆ cycloalkyl, and C₁-C₄ haloalkyl. In some embodiments, R³ is phenyl, optionally substituted with 1, 2, 3, 4, or 5 substituents independently selected from halo (e.g., F, Cl, Br). In some embodiments, R³ is phenyl, substituted with one halo (e.g., F, Cl, Br). The substitution may be at any position on the phenyl ring. In some embodiments, R³ is 4-chlorophenyl.

In some embodiments, R^{7a} and R^{7b} are each selected from C₁-C₆ alkyl, hydrogen, and halo-C₁-C₆-alkyl. In some embodiments, R^{7a} is C₁-C₆ alkyl. In some embodiments, R^{7a} is methyl. In some embodiments, R^{7b} is C₁-C₆ alkyl. In some embodiments, R^{7b} is methyl. In some embodiments, both of R^{7a} and R^{7b} are C₁-C₆ alkyl. In some embodiments, both of R^{7a} and R^{7b} are methyl.

In some embodiments, the group of formula (i) is:

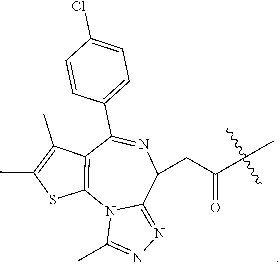 [SMILES: *C(=O)CC1N=C(c2ccc(Cl)cc2)c2c(sc(C)c2C)-n2c(C)nnc21]

The compound includes B, which is a linker. In some embodiments, B separates the bromodomain inhibitor and the GAAA repeat binding moiety by about 5 Å to about 1000 Å. In some embodiments, Bseparates the bromodomain inhibitor from the GAAA repeat by 5 Å, 10 Å, 20 Å, 50 Å, 100 Å, 150 Å, 200 Å, 300 Å, 400 Å, 500 Å, 600 Å, 700 Å, 800 Å, 900 Å, 1000 Å, or any suitable range therebetween (e.g., 5-100 Å, 50-500 Å, 150-700 Å, etc.). In some embodiments, B separates two groups by about 1-200 atoms (e.g., 1, 2, 3, 4, 5, 6, 7, 8, 9, 10, 15, 20, 25, 30, 35, 40, 45, 50, 60, 70, 80, 90, 100, 120, 140, 160, 180, 200, or any suitable ranges therebetween (e.g., 2-20, 10-50, etc.)).

B can include one or more groups selected from —C(R′)₂—, —CH═CH—, —C≡C—, —O—, —NR′—, —BR′—, —S—, —C(O)—, —C(NR′)—, —S(O)—, —S(O)₂-, arylene, heteroarylene, cycloalkylene, and heterocyclylene, wherein each R′ is independently selected from hydrogen, C₁-C₆ alkyl, C₂-C₆ alkenyl, C₂-C₆ alkynyl, aryl, arylalkyl, cycloalkyl, cycloalkylalkyl, heterocyclyl, heterocyclyl, heteroaryl, and heteroarylalkyl, and wherein each alkyl, alkenyl, alkynyl, arylene, heteroarylene, cycloalkylene, and heterocyclylene is independently unsubstituted or substituted with 1, 2, or 3 substituents. In some embodiments, B comprises a combination of one or more groups selected from —O—, —CH₂—, —C(O)—, and —NR′—.

In some embodiments, B comprises one or more —(CH₂CH₂O)— (oxyethylene) groups, e.g., 1-20 —(CH₂CH₂O)— groups (e.g., 1, 2, 3, 4, 5, 6, 7, 8, 9, 10, 11, 12, 13, 14, 15, 16, 17, 18, 19, or 20 —(CH₂CH₂O)— groups, or any range therebetween). In some embodiments, B comprises a —(CH₂CH₂O)—, —(CH₂CH₂O)₂—, —(CH₂CH₂O)₃—, —(CH₂CH₂O)₄—, —(CH₂CH₂O)₅—, —(CH₂CH₂O)₆—, —(CH₂CH₂O)₇—, —(CH₂CH₂O)₈—, —(CH₂CH₂O)₉—, or —(CH₂CH₂O)₁₀— group.

In some embodiments, B comprises one or more alkylene groups (e.g., —(CH₂)ₙ—, wherein n is 1, 2, 3, 4, 5, 6, 7, 8, 9, or 10). In some embodiments, B comprises one or more ethylene groups. In some embodiments, B comprises one or more propylene groups.

In some embodiments, B comprises at least one —C(O)NH— group. In some embodiments, B comprises at least one —C(O)N(CH₃)— group.

In some embodiments, B comprises at least one —NR═— group, where R′ is selected from hydrogen or C₁-C₆ alkyl. In some embodiments, B comprises at least one alkylamino (e.g., N(CH₃), N((CH₂)ₙCH₃), wherein n is 1, 2, 3, 4, 5, 6, 7, 8, 9, or 10). In some embodiments, B comprises at least one —NH— group.

In some embodiments, B is —NR′—(CH₂CH₂O)ₘ—(CH₂)ₚ—C(O)NR′—(CH₂)_{q}—NR′—(CH₂)ᵣ—NR′—; m, p, q, and r are each independently selected from 1, 2, 3, 4, 5, 6, 7, 8, 9, or 10; and each R′ is selected from hydrogen or C₁-C₆ alkyl. In some embodiments, m is 5, 6, 7, 8, 9, or 10. In some embodiments, m is 5, 6, or 7. In some embodiments, p, q, and r are each independently selected from 2, 3, 4, or 5. In some embodiments, one or more R′ is hydrogen. In some embodiments, each R′ is hydrogen. In some embodiments, one or more R′ is C₁-C₆ alkyl.

In select embodiments, B is —NH—(CH₂CH₂O)₆—(CH₂)₂—C(O)NH—(CH₂)₃—N(CH₃)—(CH₂)₃—NH—.

In some embodiments, the compound is selected from:

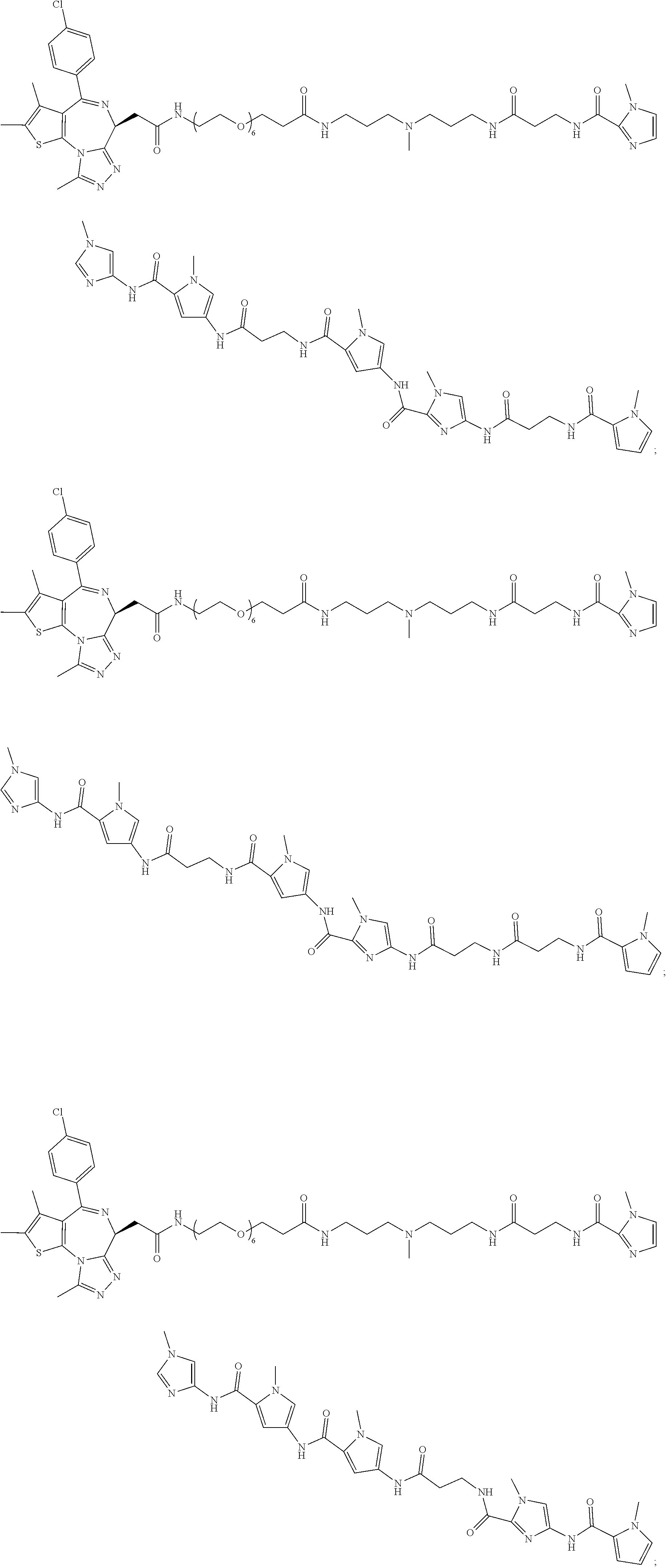

and pharmaceutically acceptable salts thereof. In some embodiments, the tertiary amine group in these compounds can be protonated, such that the compound is in the form of a pharmaceutically acceptable salt with a suitable anion, e.g., as described below.

The compounds may exist as stereoisomers wherein one or more asymmetric or chiral centers are present. Each stereocenter is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem. 1976, 45: 13-30. Various stereoisomers and mixtures thereof are specifically included within the scope of this disclosure. Stereoisomers include enantiomers and diastereomers, and mixtures of enantiomers or diastereomers. Individual stereoisomers of the compounds may be prepared synthetically from commercially available starting materials that contain asymmetric or chiral centers, or by preparation of racemic mixtures followed by methods of resolution well-known to those of ordinary skill in the art. These methods of resolution are exemplified by: (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM202JE, England; (2) direct separation of the mixture of optical enantiomers on chiral chromatographic columns; or (3) fractional recrystallization methods.

It should be understood that the compounds may exist in different tautomeric forms, and all such forms are included within the scope of the disclosure.

The present disclosure also includes an isotopically-labeled compound, which is identical to those recited in formula (I), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium (²H) can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

Compounds disclosed herein can exist in solvated as well as unsolvated forms with pharmaceutically acceptable solvents such as water, ethanol, and the like, and it is intended that the disclosure encompass both solvated and unsolvated forms. In one embodiment, the compound is amorphous. In one embodiment, the compound is a single polymorph. In another embodiment, the compound is a mixture of polymorphs. In another embodiment, the compound is in a crystalline form.

a. Pharmaceutically Acceptable Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric, and the like. Amino groups of the compounds may also be quaternized with alkyl chlorides, bromides, and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethyl aniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

b. Methods of Synthesis

In another aspect, disclosed herein are methods for making disclosed compounds, or a pharmaceutically acceptable salt thereof. Broadly, the disclosed compounds and pharmaceutically acceptable salts thereof can be prepared by any process known to be applicable to the preparation of chemically related compounds. Exemplary suitable synthetic schemes are provided in the Examples section.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for example in “Vogel's Textbook of Practical Organic Chemistry,” 5th edition (1989), by Furniss, Hannaford, Smith, and Tatchell, pub. Longman Scientific & Technical, Essex CM20 2JE, England.

Reaction conditions and reaction times for each individual step can vary depending on the particular reactants employed and substituents present in the reactants used. Reactions can be worked up in a conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method, are included in the scope of the disclosure. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006).

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as, for example, chromatographic separation, recrystallization, or enzymatic resolution).

Similarly, when a pure geometric isomer of a compound is required, it can be obtained by carrying out one of the procedures described herein using a pure geometric isomer as a starting material, or by resolution of a mixture of the geometric isomers of the compound or intermediates using a standard procedure such as chromatographic separation.

The synthetic schemes and specific examples as described are illustrative and are not to be read as limiting the scope of the disclosure or the claims. Alternatives, modifications, and equivalents of the synthetic methods and specific examples are contemplated.

### Pharmaceutical Compositions

The disclosed compounds may be incorporated into pharmaceutical compositions suitable for administration to a subject (such as a patient, which may be a human or non-human). The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the disclosure are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease or condition, the prophylactically effective amount will be less than the therapeutically effective amount.

The pharmaceutical compositions may include pharmaceutically acceptable carriers. The term “pharmaceutically acceptable carrier,” as used herein, means a non-toxic, inert solid, semi-solid or liquid filler, diluent, encapsulating material, or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral injections) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90% by weight of the composition.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10% by weight of the composition.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50% by weight of the composition.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10% by weight of the composition.

Suitable colorants include a colorant such as an FD&C dye. When used, the amount of colorant in a systemic or topical composition is typically about 0.005 to about 0.1% by weight of the composition.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s), when used, in a systemic or topical composition is typically about 0.001 to about 1% by weight of the composition.

Suitable antioxidants include butylated hydroxyani sole (“BHA”), butylated hydroxytoluene (“BHT”), and vitamin E. The amount of antioxidant(s) in a systemic or topical composition is typically about 0.1 to about 5% by weight of the composition.

Suitable preservatives include benzalkonium chloride, methyl paraben, and sodium benzoate. The amount of preservative(s) in a systemic or topical composition is typically about 0.01 to about 5% by weight of the composition.

Suitable glidants include silicon dioxide. The amount of glidant(s) in a systemic or topical composition is typically about 1 to about 5% by weight of the composition.

Suitable solvents include water, isotonic saline, ethyl oleate, glycerin, hydroxylated castor oils, alcohols such as ethanol, and phosphate buffer solutions. The amount of solvent(s) in a systemic or topical composition is typically from about 0 to about 100% by weight of the composition.

Suitable suspending agents include AVICEL RC-591 (from FMC Corporation of Philadelphia, PA) and sodium alginate. The amount of suspending agent(s) in a systemic or topical composition is typically about 1 to about 8% by weight of the composition.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5% by weight of the composition.

Although the amounts of components in the systemic compositions may vary depending on the type of systemic composition prepared, in general, systemic compositions include 0.01% to 50% by weight of an active compound and 50% to 99.99% by weight of one or more carriers. Compositions for parenteral administration typically include 0.1% to 10% by weight of actives and 90% to 99.9% by weight of a carrier including a diluent and a solvent.

Compositions for oral administration can have various dosage forms. For example, solid forms include tablets, capsules, granules, and bulk powders. These oral dosage forms include a safe and effective amount, usually at least about 5% by weight, and more particularly from about 25% to about 50% by weight of actives. The oral dosage compositions include about 50% to about 95% by weight of carriers, and more particularly, from about 50% to about 75% by weight.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose, and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound as disclosed herein), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this disclosure.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol, and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound as disclosed herein), or a pharmaceutically acceptable salt thereof), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this disclosure are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95% by weight of the composition.

Suitable propellants include propane, butane, isobutane, dimethyl ether, carbon dioxide, nitrous oxide, and combinations thereof. The amount of propellant(s) in a topical composition is typically about 0% to about 95% by weight of the composition.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95% by weight of the composition.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95% by weight of the composition.

The amount of thickener(s) in a topical composition is typically about 0% to about 95% by weight of the composition.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95% by weight of the composition.

The amount of fragrance in a topical composition is typically about 0% to about 0.5%, particularly, about 0.001% to about 0.1% by weight of the composition.

Suitable pH adjusting additives include HCl or NaOH in amounts sufficient to adjust the pH of a topical pharmaceutical composition.

### Methods of Use

The disclosed compounds and pharmaceutical compositions may be used in methods for treatment of diseases or disorders, such as a disease or disorder characterized by having expansion of GAAA repeats, e.g., in a disease-related gene. In some embodiments, the disclosed compounds and pharmaceutical compositions are useful in methods of treating proliferative disorders, e.g., cancers.

Accordingly, in some embodiments, disclosed herein are methods of treating a disease or disorder in a subject in need thereof, comprising administering to the subject a therapeutically effective amount of a compound disclosed herein, or a pharmaceutically acceptable salt thereof, or a pharmaceutical composition comprising a compound disclosed herein, or a pharmaceutically acceptable salt thereof.

In some embodiments, the disease or disorder is a proliferative disease or disorder, e.g., a disease or disorder that occurs due to abnormal growth or extension by the multiplication or replication of cells. Proliferative diseases or disorders may include benign, premalignant, and malignant cell proliferation. In some embodiments, the proliferative disease is cancer. The term “cancer” refers to a class of diseases characterized by development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. In some embodiments, the proliferative disease or disorder includes neovascularization associated with tumor angiogenesis, macular degeneration (e.g., wet/dry age related macular degeneration), corneal neovascularization, diabetic retinopathy, neovascular glaucoma, myopic degeneration. In some embodiments, the proliferative disease or disorder includes restenosis and polycystic kidney disease.

In some embodiments, the compounds and pharmaceutical compositions disclosed herein are used for treating cancer in a subject in need thereof In some embodiments, the cancer comprises a solid tumor. In some embodiments, the cancer is metastatic cancer. In some embodiments, the disclosed compounds, compositions, or methods result in suppression of elimination of metastasis. In some embodiments, the disclosed compounds, compositions, or methods result in decreased tumor growth. In some embodiments, the disclosed compounds, compositions, or methods prevent tumor recurrence.

The disclosed compounds may be useful to treat a wide variety of cancers including carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, lymph nodes, muscle tissue, ovary, pancreas, prostate, skin, spleen, stomach, testicle, thyroid, or uterus. The cancer may be a primary or secondary cancer in that it can be located where it originated or metastasis from cancer in other organs, respectively. In some embodiments, the cancer is kidney cancer, liver cancer, prostate cancer, or ovarian cancer.

In some embodiments, the cancer comprises cancer of the kidney. Exemplary types of kidney cancer include: renal cell carcinoma (e.g., clear cell renal cell carcinoma, papillary renal cell carcinoma, chromophobe renal cell carcinoma, collecting duct renal cell carcinoma, multilocular cystic renal cell carcinoma, medullary carcinoma, mucinous tubular and spindle cell carcinoma, neuroblastoma-associated renal cell carcinoma); transitional cell carcinoma (also known as urothelial carcinoma); renal pelvis carcinoma; Wilms tumor (nephroblastoma); renal sarcoma; angiomyolipoma; and oncocytoma.

In some embodiments, the cancer comprises cancer of the ovaries. Ovarian cancers comprise epithelial ovarian cancer, germ cell ovarian tumors (e.g., teratoma, dysgerminomas, endodermal sinus tumors, and choriocarcinomas), sex cord stromal tumors, ovarian cysts, and borderline ovarian tumors. The ovarian cancer may further comprise primary peritoneal cancer and fallopian tube cancer.

In some embodiments, the cancer comprises cancer of the liver. Exemplary types of liver cancer include: hepatocellular carcinoma (HCC), cholangiocarcinoma, and hepatoblastoma. In some embodiments, the liver cancer is secondary liver cancer, e.g., a cancer that starts originates elsewhere in the body, such as the colon, lung, or breast, and then spreads to the liver. Liver cancer can also form from other structures within the liver such as the bile duct, blood vessels and immune cells.

In some embodiments, the cancer comprises cancer of the prostate. Almost all prostate cancers are adenocarcinomas which develop from the gland cells of the prostate. Rare forms of prostate cancer include sarcomas, small cell carcinomas, neuroendocrine tumors (other than small cell carcinomas) or transitional cell carcinomas. The prostate cancer may be any of Gleason Grades 1-5. The “Gleason Grade” is the most commonly use prostate cancer grading system. It involves assigning numbers to cancerous prostate tissue, ranging from 1 through 5, based on how much the arrangement of the cancer cells mimics the way normal prostate cells form glands. The prostate cancer may be prostate-specific antigen (PSA), prostate stem cell antigen (PSCA) or prostate-specific membrane antigen (PSMA) positive.

In the methods of treatment disclosed herein, a compound or pharmaceutical composition may be administered to the subject by any convenient route of administration, whether systemically/peripherally or at the site of desired action, including but not limited to, oral (e.g., by ingestion); topical (including e.g. transdermal, intranasal, ocular, buccal, and sublingual); pulmonary (e.g., by inhalation or insufflation therapy using, e.g., an aerosol, e.g., through mouth or nose); rectal; vaginal; parenteral (e.g., by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal injection); or by implant of a depot, for example, subcutaneously or intramuscularly. In some embodiments, the administration comprises oral administration. In some embodiments, the administration comprises parenteral administration. In some embodiments, the administration comprises intratumoral administration. Additional modes of administration may include adding the compound and/or a composition comprising the compound to a food or beverage, including a water supply for an animal, to supply the compound as part of the animal's diet.

It will be appreciated that appropriate dosages of the compounds, and compositions comprising the compounds, can vary from patient to patient. Determining the optimal dosage will generally involve the balancing of the level of therapeutic benefit against any risk or deleterious side effects of the treatments of the present disclosure. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. In general, a suitable dose of the compound is in the range of about 100 μg to about 250 mg per kilogram body weight of the subject per day.

The compound or composition may be administered once, on a continuous basis (e.g., by an intravenous drip), or on a periodic/intermittent basis, including about once per hour, about once per two hours, about once per four hours, about once per eight hours, about once per twelve hours, about once per day, about once per two days, about once per three days, about twice per week, about once per week, and about once per month. The composition may be administered until a desired reduction of symptoms is achieved.

A compound described herein may be used in combination with other known therapies. Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A compound or composition described herein and the at least one additional therapeutic agent can be administered simultaneously, in the same or in separate compositions, or sequentially. For sequential administration, the compound described herein can be administered first, and the additional agent can be administered subsequently, or the order of administration can be reversed.

In some embodiments, the compound described herein is administered with at least one additional therapeutic agent, such as a chemotherapeutic agent. In certain embodiments, the compound described herein is administered in combination with one or more additional chemotherapeutic agents.

The chemotherapeutic agent may be a chemotherapeutic agent identified on the “A to Z List of Cancer Drugs” published by the National Cancer Institute. Chemotherapeutics include, but are not limited to, cyclophosphamide, methotrexate, 5-fluorouracil, doxorubicin, docetaxel, daunorubicin, bleomycin, vinblastine, dacarbazine, cisplatin, paclitaxel, raloxifene hydrochloride, tamoxifen citrate, abemacicilib, afinitor (Everolimus), alpelisib, anastrozole, pamidronate, anastrozole, exemestane, capecitabine, epirubicin hydrochloride, eribulin mesylate, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, and tucatinib.

In some embodiments, a compound described herein is administered in combination with other therapeutic treatment modalities, including surgery (e.g., surgical resection), percutaneous ablation, radiation, transplantation (e.g., stem cell transplantation, bone marrow transplantation), cryotherapy, immunotherapy, chemoembolisation, hormone therapy, and/or thermotherapy. Such combination therapies may allow for lower dosages of the administered agent and/or other chemotherapeutic agent, thus avoiding possible toxicities or complications associated with the various therapies.

In some embodiments, the second therapy includes immunotherapy. Immunotherapies include chimeric antigen receptor (CAR) T-cell or T-cell transfer therapies, cytokine therapy, immunomodulators, cancer vaccines, or administration of antibodies (e.g., monoclonal antibodies).

In some embodiments, the immunotherapy comprises administration of antibodies. The antibodies may target antigens either specifically expressed by tumor cells or antigens shared with normal cells. In some embodiments, the immunotherapy may comprise an antibody targeting, for example, CD20, CD33, CD52, CD30, HER (also referred to as erbB or EGFR), VEGF, CTLA-4 (also referred to as CD152), epithelial cell adhesion molecule (EpCAM, also referred to as CD326), and PD-1/PD-L1. Suitable antibodies include, but are not limited to, rituximab, blinatumomab, trastuzumab, gemtuzumab, alemtuzumab, ibritumomab, tositumomab, bevacizumab, cetuximab, panitumumab, ofatumumab, ipilimumab, brentuximab, pertuzumab and the like). In some embodiments, the additional therapeutic agent may comprise anti-PD-1/PD-L1 antibodies, including, but not limited to, pembrolizumab, nivolumab, cemiplimab, atezolizumab, avelumab, durvalumab, and ipilimumab. The antibodies may also be linked to a chemotherapeutic agent. Thus, in some embodiments, the antibody is an antibody-drug conjugate.

The immunotherapy (e.g., administration of antibodies) may be administered to a subject by a variety of methods. In any of the uses or methods described herein, administration may be by various routes known to those skilled in the art, including without limitation oral, inhalation, intravenous, intramuscular, topical, subcutaneous, systemic, and/or intraperitoneal administration to a subject in need thereof. The immunotherapy may be administered by parenteral administration (including, but not limited to, subcutaneous, intramuscular, intravenous, intraperitoneal, intracardiac and intraarticular injections).

### p Kits

Compounds and/or compositions disclosed herein may be assembled into kits or pharmaceutical systems. Kits or pharmaceutical systems according may include a carrier or package such as a box, carton, tube, or the like, having in close confinement therein one or more containers, such as vials, tubes, ampoules, or bottles, which contain a compound disclosed herein or a pharmaceutically acceptable salt thereof, or a pharmaceutical composition comprising a compound disclosed herein or a pharmaceutically acceptable salt thereof.

The kits can also comprise other agents and/or products co-packaged, co-formulated, and/or co-delivered with other components. For example, a drug manufacturer, a drug reseller, a physician, a compounding shop, or a pharmacist can provide a kit comprising a disclosed compound and/or product and another agent (e.g., a chemotherapeutic, a monoclonal antibody, a pain reliever, an anti-seizure medicine, a steroid, an anti-emetic) for delivery to a patient. Individual member components of the kits may be physically packaged together or separately.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein.

The following examples further illustrate aspects of the disclosure, but should not be construed as in any way limiting its scope.

### EXAMPLES

### Example 1

### Compound Syntheses

The compounds disclosed herein were synthesized using standard solid-phase peptide synthesis techniques and amide coupling reactions, as shown below in Schemes 1 and 2.

First, tert-butyl (S)-2-(4-(4-chlorophenyl)-2,3,9-trimethyl-6H-thieno[3,2-f][1,2,4]triazolo[4,3-a][1,4]diazepin-6-yl)acetate was deprotected with formic acid to yield (S)-2-(4-(4-chlorophenyl)-2,3,9-trimethyl-6H-thieno[3,2-f][1,2,4]triazolo[4,3-a][1,4]diazepin-6-yl)acetic acid (“JQ1 acid”).

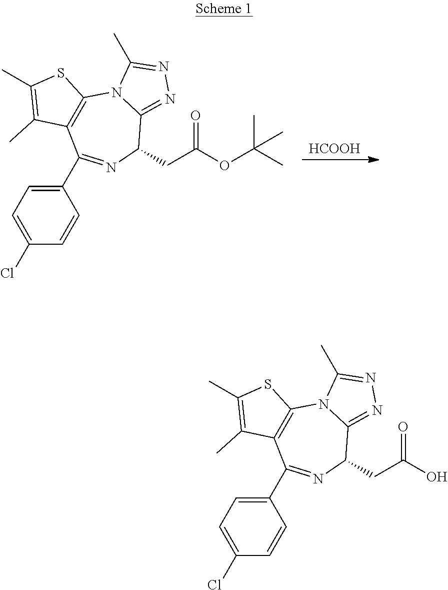

The GAAA-targeting polyamide portion of the molecule was prepared using standard fluorenylmethoxycarbonyl (Fmoc) solid phase peptide synthesis techniques using suitably protected building blocks. Once the polyamide was synthesized, it was cleaved from the resin using 3,3′-diamino-N-methyldipropylamine. As shown in Scheme 2, the product can be coupled to H₂N—(CH₂CH₂O)₆—CH₂CH₂COOH using 1-[bis(dimethylamino)methylene]-1H-1,2,3-triazolo[4,5-b]pyridinium 3-oxid hexafluorophosphate (HATU), followed by final coupling to the JQ1 acid using HATU. Alternatively, H₂N—(CH₂CH₂O)₆—CH₂CH₂COOH can first be coupled to the JQ1 acid using HATU, and that product can then be coupled to the polyamide-containing compound. In each case, the final products were purified to a minimum of 95% purity. HPLC conditions for chemical characterization: 1.0 mL/min, Solvent A: 0.1% trifluoroacetic acid (TFA) in H₂O, Solvent B: 0.075% TFA in acetonitrile, Gemini, Column: C18 5 μm 110A 150*4.6 mm.

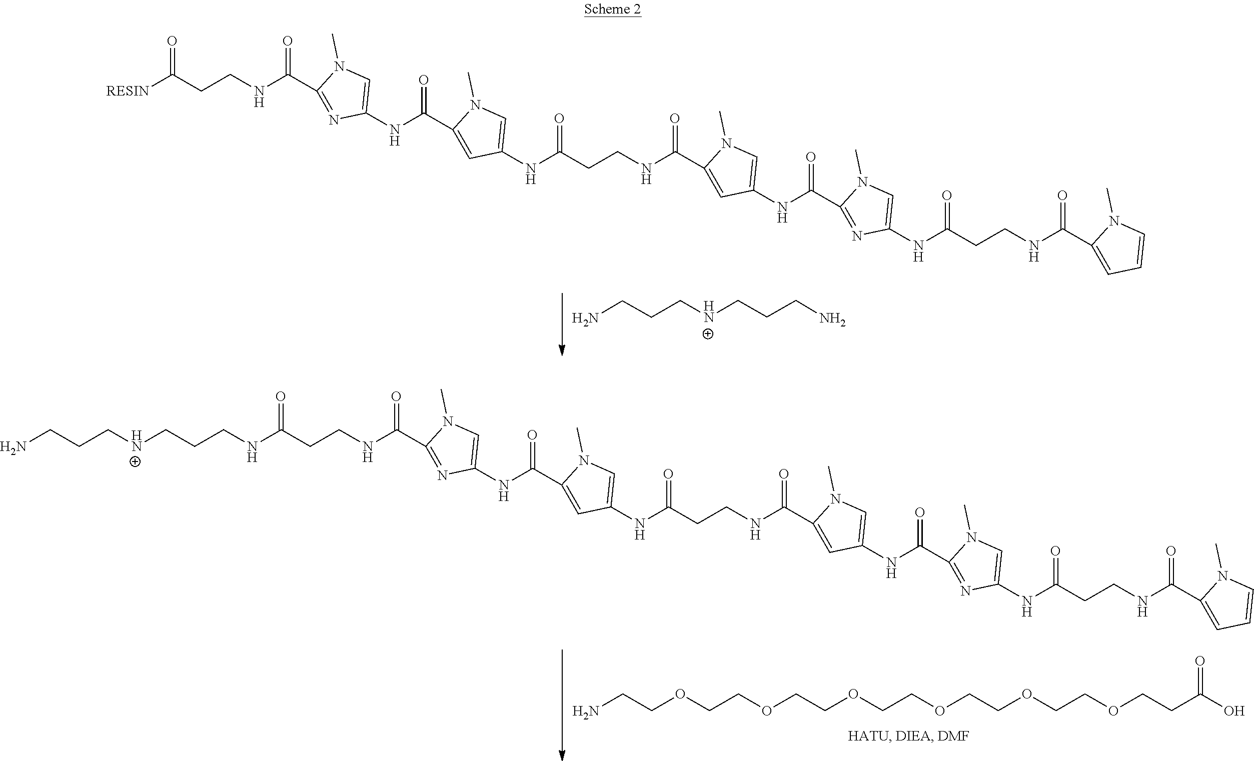

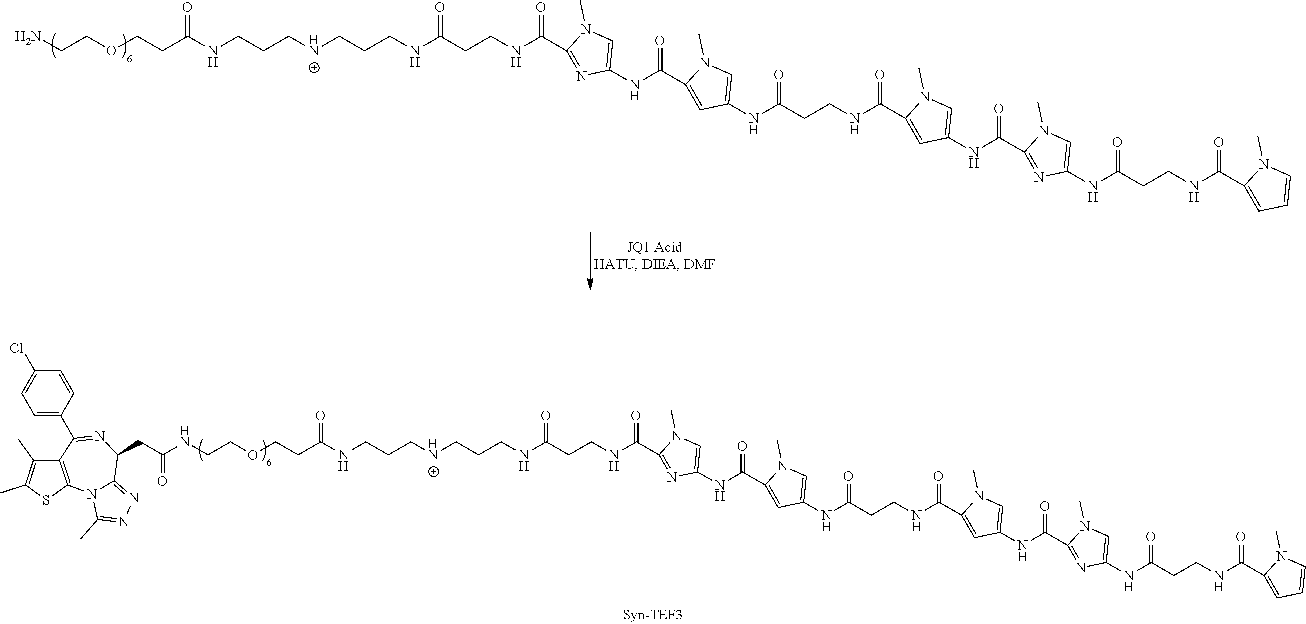

Additional compounds, described herein as Syn-TEF1, Syn-TEF2, and Syn-TEF4, were prepared similarly. Syn-TEF1 and SynTEF-2 were designed to target GAAA repeats, while Syn-TEF4 is a control compound designed to target GGAA repeats.

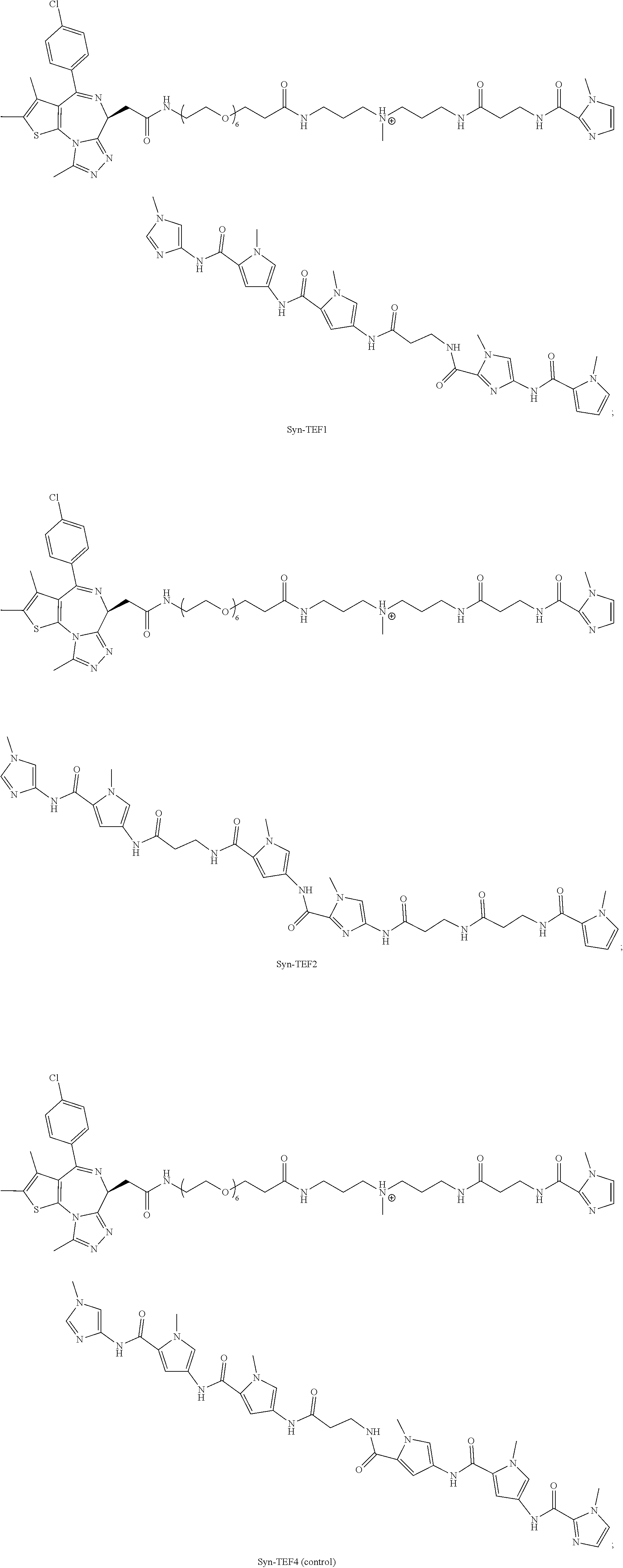

LC-MS characterization data for these compounds is provided in Table 1.

TABLE 1

| Compound | LCMS |
|---|---|
| Syn-TEF1 | 1602.9 |
| Syn-TEF2 | 1746 |
| Syn-TEF3 | 1674 |
| Syn-TEF4 (control) | 1605.9 |

### Example 2

### Recurrent Repeat Expansions (rREs) in Cancer

Uniformly-processed alignments of whole-genome sequencing data were collected from the International Cancer Genome Consortium (ICGC) and The Cancer Genome Atlas (TCGA) pan-cancer analysis of whole genomes (PCAWG) dataset. After filtering, these data consist of 2,622 cancer genomes from 2509 patients across 29 different cancer types. Each cancer type was treated as its own cohort and analyzed independent of other cancer types. Because variations in repeat length are common, the PCAWG dataset was particularly valuable to distinguish recurrent somatic events from natural genetic variation. rREs were identified with Expansion Hunter De Novo (EHdn), which measures TRs whose length exceeds the sequencing read length in short-read sequencing datasets. EHdn has been validated with both known and novel repeat expansions by several independent groups. After running EHdn across all 2,622 genomes, for each TR locus a non-parametric statistical test was applied to determine whether repeat length is longer in tumor genomes compared to matching normal genomes. Using this method, 285,363 TRs and identified 578 candidate rREs with EHdn (locus-level false discovery rate (FDR) <10%) were analyzed.

The candidate rREs were validated with independent cohorts of matching tumor-normal tissue samples for breast, prostate, and kidney cancer (15, 18, and 12 patients, respectively). The ability to detect candidate rREs in independent cohorts of samples 31%. One explanation for this lower detection rate is that cancer genomes may be more complicated than genomes of patients with monogenic pathogenic repeat expansions. Cancer genomes are more likely to contain chromosomal amplifications and extrachromosomal circular DNAs, which alter the read depth in the local vicinity. These genetic alterations will affect read counts and TR expansion calls, which is not an issue for germline expansions in neurodegenerative diseases.

To account for copy number variants, a local read depth filtering method was devised and implemented, which normalized the signal originating from repeat reads using the read depth in the vicinity of the STR. The filtering approach removed >75% (418/578) false-positive candidate rREs. Several rRE candidates that were removed were situated in hotspots for chromosomal amplification, such as chromosomal 8q amplifications that increase MYC production in breast cancer; these loci may be due to amplification rather than repeat expansions and thus their removal is important.

To determine if this approach improved rRE identification, 28 loci from the tumor-normal pairs for 12-18 breast, prostate, and kidney cancer samples were studied by PCR and gel electrophoresis. Of the 14 rREs that passed local read depth filtering, 57% (8/14 loci) were validated in the independent cohorts. The loci unable to be validated had lower expansion frequencies (5-12%). Of the 14 candidate rREs that failed the local read depth filter, 29% (4/14) were detected in independent cohorts of samples indicating that the filtering removes most loci that cannot be validated, but also removed some true positives as well.

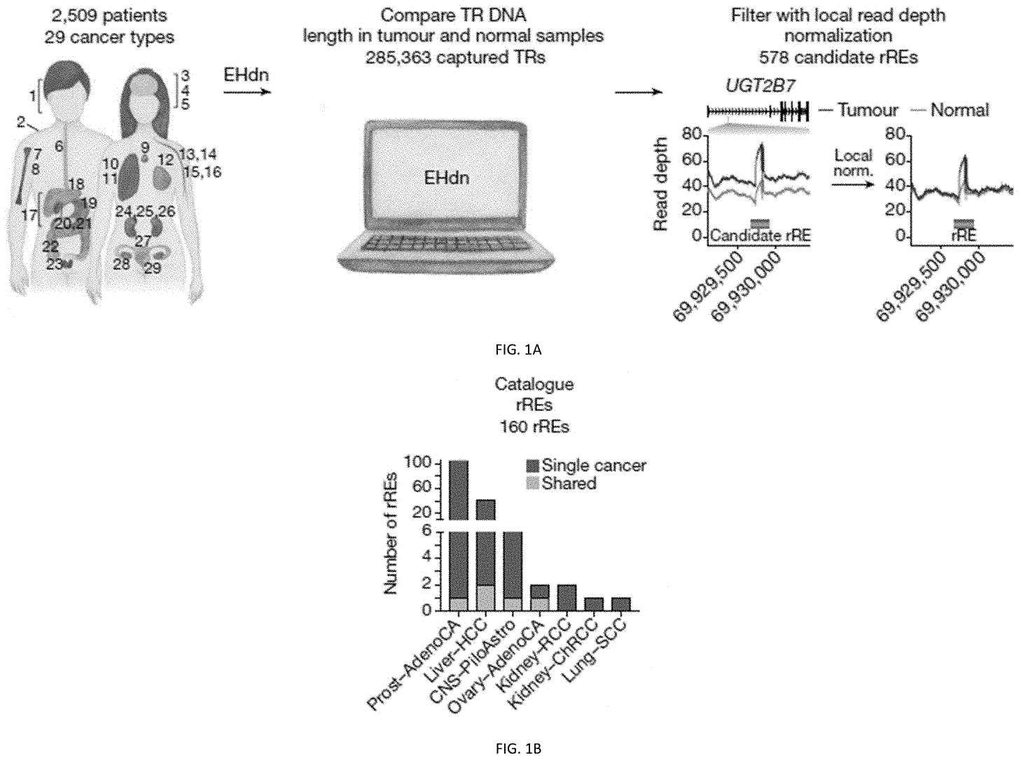

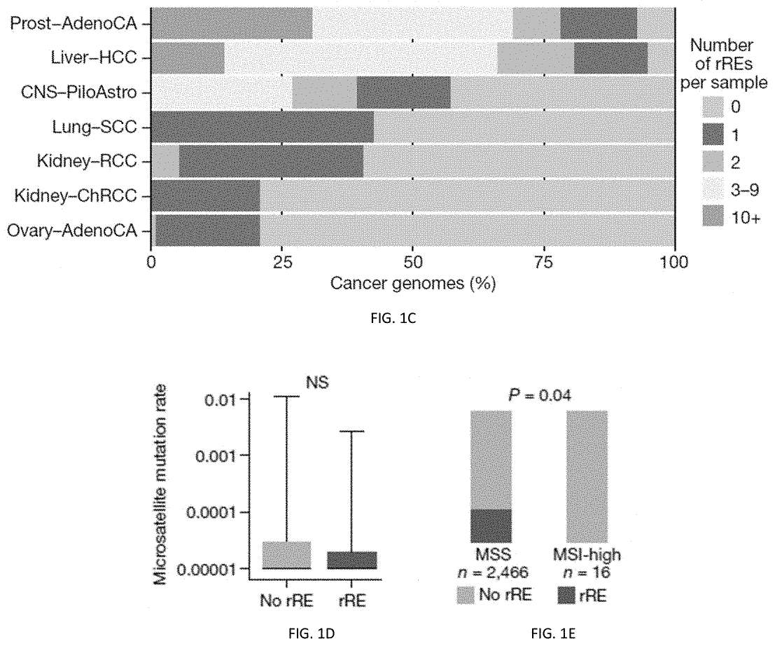

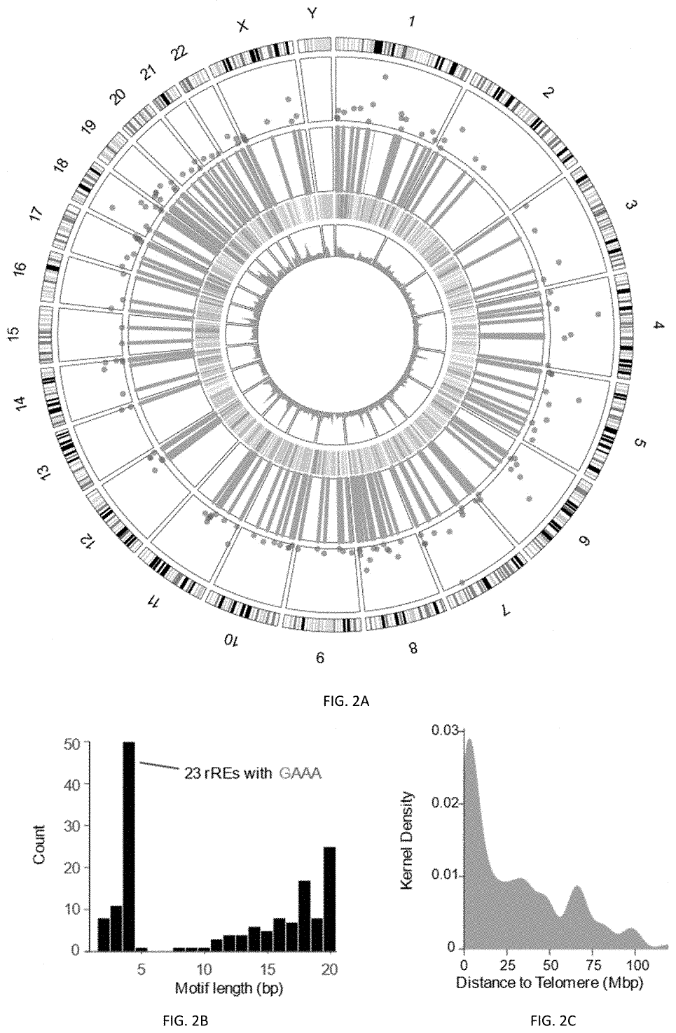

After accounting for local read depth, 160 rREs were detected in 7 human cancers (FIG. 1B). 147 of the 160 rREs were successfully annotated and the expansions in 134 (91%) were confirmed with another repeat expansion detection algorithm, ExpansionHunter. Most rREs were in prostate and liver cancer, but rREs were also detected in ovarian, pilocytic astrocytoma, renal cell carcinoma, chromophobe renal cell carcinoma, and lung squamous cell carcinoma. Thus, rREs are found in tissues derived from each of the three primary germ layers (ectoderm, mesoderm, and endoderm). In prostate and liver cancer, most cancer genomes (93% and 95%, respectively) contain at least one rRE, with some genomes harboring several rREs (FIG. 1C). Overall, rREs are found in 7 of 29 human cancers examined and are largely cancer subtype-specific.

No significant difference in STR mutation rate was observed for genomes with an rRE compared to those lacking a rRE (two-tailed Wilcoxon rank sum test, P=0.27, FIG. 1D). No enrichment in MSI was observed for samples harboring a rRE, but a small but significant preference was found for rREs in MSS samples (Two-tailed Wilcoxon rank-sum test, P=0.04, FIG. 1E). There was no significant correlation between MSI cancers and the percentage of patients that have an rRE (R²=0.04). Little correlation was observed between the percentage of MSI cancers in a particular cancer type and the number of repeat expansions identified (R²=0.07). Thus, the data are consistent with a model where rREs are formed by a process that is distinct from MSI.

A multiple linear regression was performed to predict the number of rREs in a sample based on single base substitution (SBS) and doublet base substitution (DBS) signatures, respectively. The best predictors were selected using best subset selection and age was controlled for as a possible confounding factor by including it in the selection process. None of the SBS signatures showed a statistically significant association with rREs, and only one DBS signature, DB S2, showed a very weak association with rREs (R²=0.12). When the Lung-SCC data were removed, this weak association was no longer observed. Taken together, the data suggested that rREs appear to arise from mutation events that are independent of known cancer mutational signatures.

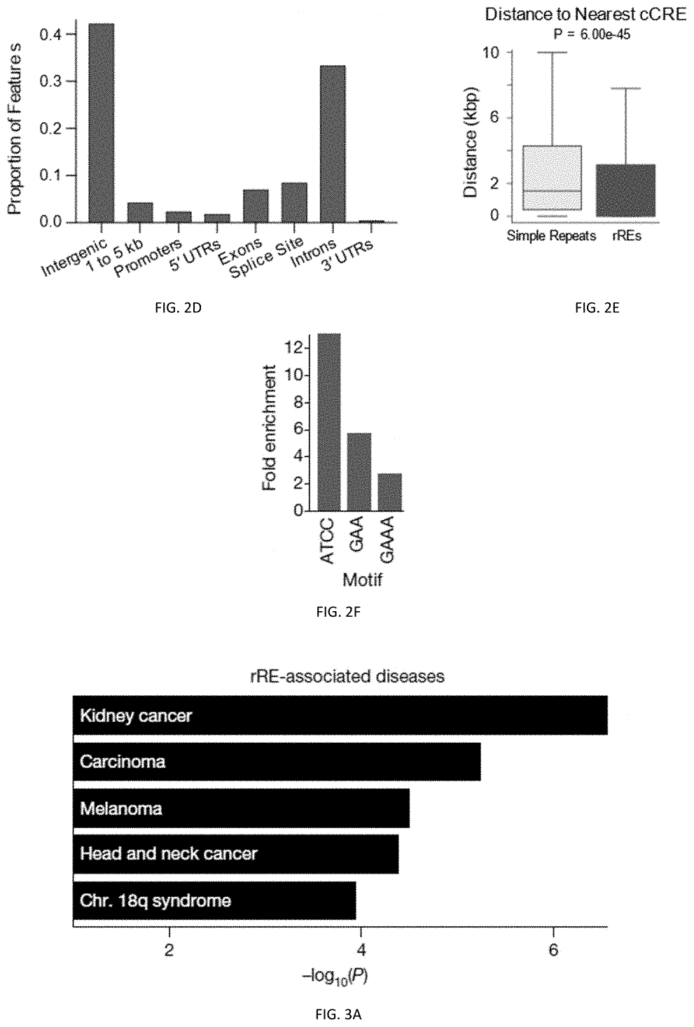

Genome-wide characteristics of the rREs were examined (FIG. 2A). rREs tended to occur in late replicating regions, but this trend was not statistically significant when compared to the control catalog of simple sequence repeats. Among the 160 rREs, a variety of different motifs were observed (Table 2), whose repeat unit length follows a bimodal distribution, consistent with REs identified in other diseases (FIG. 2B). rREs were distributed across a range of GC content; approximately half (76/160) have GC content less than 50%. Six rREs contained a known pathogenic motif, all of which were GAA. It was examined whether any motifs were enriched in the rRE catalogue as compared with the Tandem Repeat Finder (TRF) catalogue. Although this enrichment could arise from a biological and/or technical process, one of the three enriched motifs was GAA (FIG. 2F).

rREs were non-uniformly distributed across the genome, with a bias towards the ends of chromosome arms (FIG. 2C). The distribution of rREs relative to gene features was examined with annotatr (FIG. 2D). Several (7%) rREs labeled as exonic appeared proximal to, but not within, exons, but others were in introns, untranslated regions (UTRs), and splice sites. These results suggest rREs may play different functional roles in the regulation of gene expression.

The distance between rREs and the ENCODE candidate cis-regulatory element (cCRE) list was also measured. rREs were located closer to ENCODE cCREs than expected by chance, and 47 of 160 rREs directly overlapped with a known cCRE (Welch's t-test, P=6.00e-45, FIG. 2E).

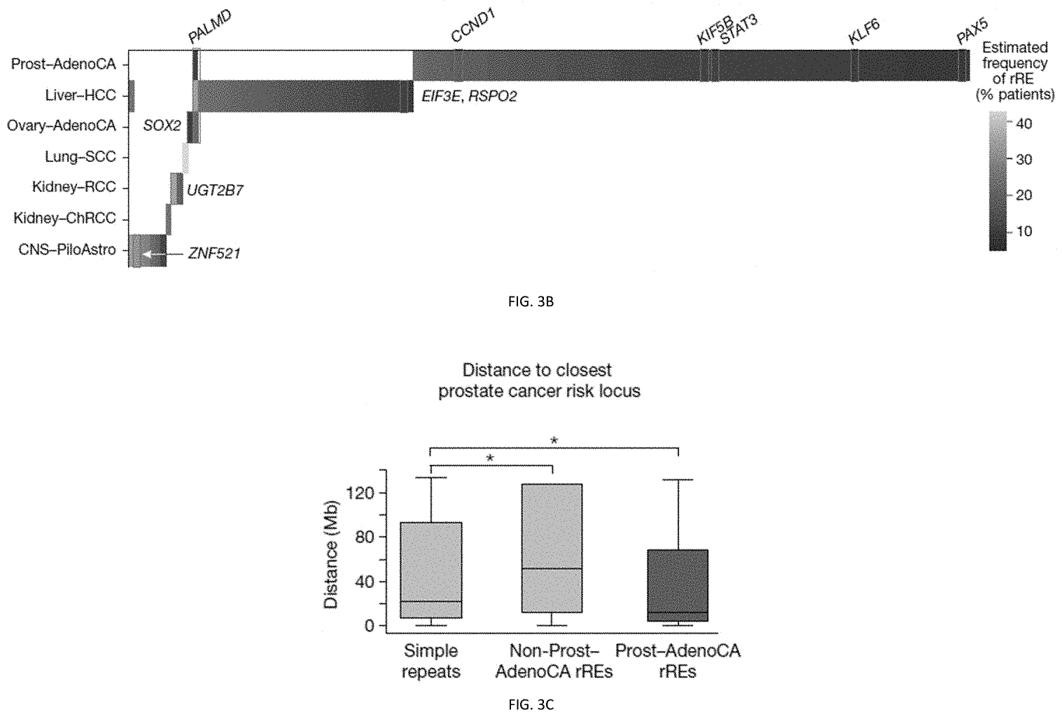

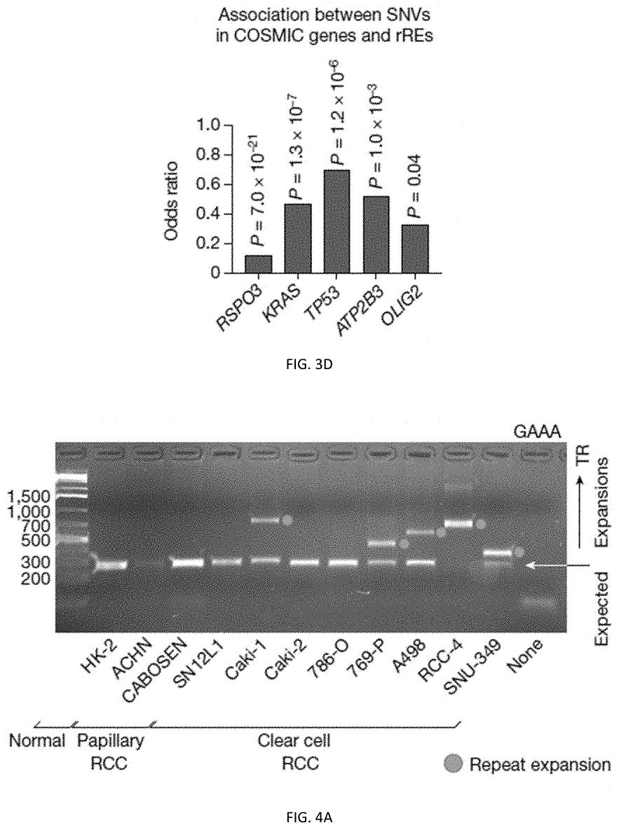

Each rRE was mapped to the nearest gene, and nine rREs mapped to Tier 1 genes present in the census of somatic mutations in cancer (COSMIC) database (FIG. 3A, Table 2). A strong correlation was observed between these rRE-associated genes and genes associated with cancer when examined for known human diseases (Jensen disease-gene associations). Indeed, of the top five diseases associated with the collection of 160 rRE genes, four were cancers (FIG. 3B). Thus, rREs are associated with genes implicated in human cancers.

Given the large number of rREs identified in prostate cancer and available data from a recent genome-wide association study that identified 63 loci associated with susceptibility to prostate cancer, the distance of rREs in prostate cancer to these risk loci was measured and it was found that rREs are located closer to prostate cancer susceptibility loci than expected by chance from a standard STR catalog (Student's t-test, FDR q=0.08, FIG. 3C).

The relationship between the occurrence of COSMIC genes and the occurrence of rREs was also examined (FIG. 3D). Interestingly, after correcting for multiple-hypothesis testing, somatic mutations were found to occur significantly more in patients' genomes without rREs for five COSMIC genes.

rREs were also correlated with evidence of cytotoxic activity. Expression of GZAIA and PRF 1 is a surrogate for the amount of infiltration of cytotoxic CD8+ T cells into tumors. This analysis is particularly interesting because MSI-high cancers often respond to immunotherapy and are often correlated with higher levels of immune cell infiltration. It is possible that some rREs may also be prognostic for immune cell infiltration. Cytotoxic activity was calculated for rREs observed in the two cancer types where there was matching gene expression (ovarian cancer and renal cell carcinoma), but a correlation was not observed between cytotoxic activity and the presence of an rRE.

The data identified (i) 160 rREs in 7 human cancers and revealed that (ii) most (155 of 160) rREs are cancer subtype specific; (iii) amongst diseases, rREs are enriched in human cancer loci; (iv) recurrent repeat expansions do not correlate with MSI status; and (v) many rREs occur near regulatory elements where they could alter gene expression.

### Example 3

### GAAA Repeat Expansion and Cancer

One recurrent repeat expansion, a GAAA repeat expansion, was identified in 3 cancer types (prostate cancer, hepatocellular carcinoma, and ovarian cancer, FIG. 3A). This repeat expansion localized to the intron of the palmdelphin gene, PALMD, which is a target of p53 and plays a role in cell death. Upon DNA damage, PALMD accumulates in the nucleus of the cell and promotes apoptosis.

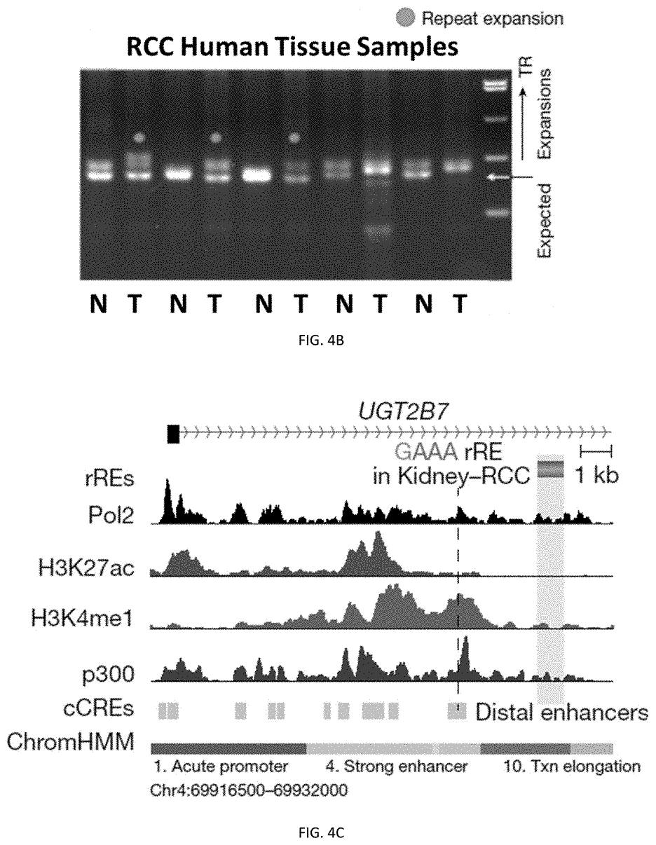

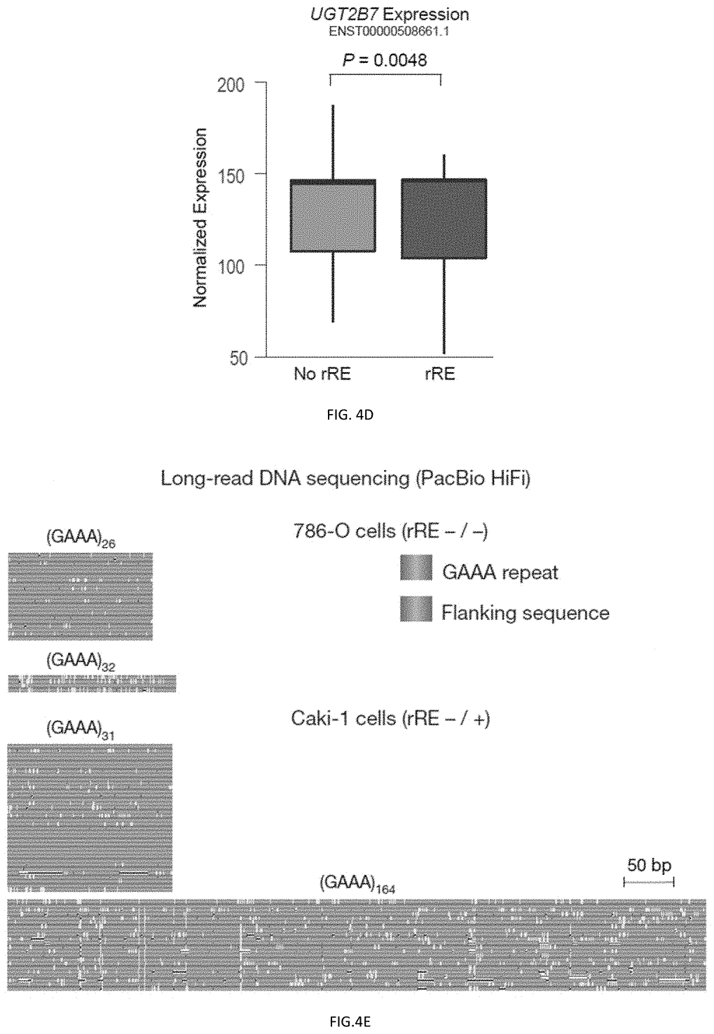

The GAAA motif located in the intron of UGT2B7 was observed in 34% of renal cell carcinoma (RCC) samples analyzed. UGT2B7 is a glucuronidase that clears small molecules—including chemotherapeutics—from the body and is selectively expressed in the kidney and liver. To further characterize, 10 kidney cell lines, including 8 from clear cell RCC, were obtained, which accounts for 90% of kidney cancer cases. Using PCR analysis and gel electrophoresis, the expected TR size of ˜26 GAAA repeats was observed in the normal kidney cell line, HK-2 (FIG. 4A). In contrast, an expansion to between ˜63 and ˜143 GAAA repeats in length was identified in 5 of 8 clear cell RCC cell lines. Most expansions were heterozygous, but one cell line, RCC-4, appeared to contain a homozygous repeat expansion with ˜131 GAAA repeats (FIG. 4A). This analysis was performed in duplicate and observed both times. Sanger DNA sequencing confirmed that these bands were GAAA repeat expansions originating from the UGT2B7 locus. Long-read DNA sequencing with highly accurate PacBio HiFi reads confirmed the PCR results and showed the precise structure of this repeat expansion at single-base-pair resolution for both the 786-O and Caki-1 cell lines (FIG. 4E).

An independent cohort of tissue samples from patients with clear cell RCC was analyzed for the presence of the UGT2B7 intronic repeat expansion. This repeat expansion was detected in five out of 12 samples (FIG. 4B) and showed more heterogeneity than the RCC cell lines, as expected for human tumor samples, rather than the clonal cell lines.

Analysis of the chromatin environment surrounding the rRE in UGT2B7 using ENCODE data revealed a nearby enhancer (two kilobases upstream), raising the possibility that this rRE alters the expression of UGT2B7 (FIG. 4C). A comparison of the gene expression between RCC samples that contained (18 samples) or lacked (31 samples) the repeat expansion, revealed a modest decrease in expression of UGT2B7 associated with the rRE, although this trend was not statistically significant. However, surprisingly, this intronic rRE is associated with a significant decrease in transcript isoform usage in UGT2B7 (Wald test with FDR correction, P=0.0048) (FIG. 4D). These results suggest a functional role for the UGT2B7 rRE in transcript usage and survival.

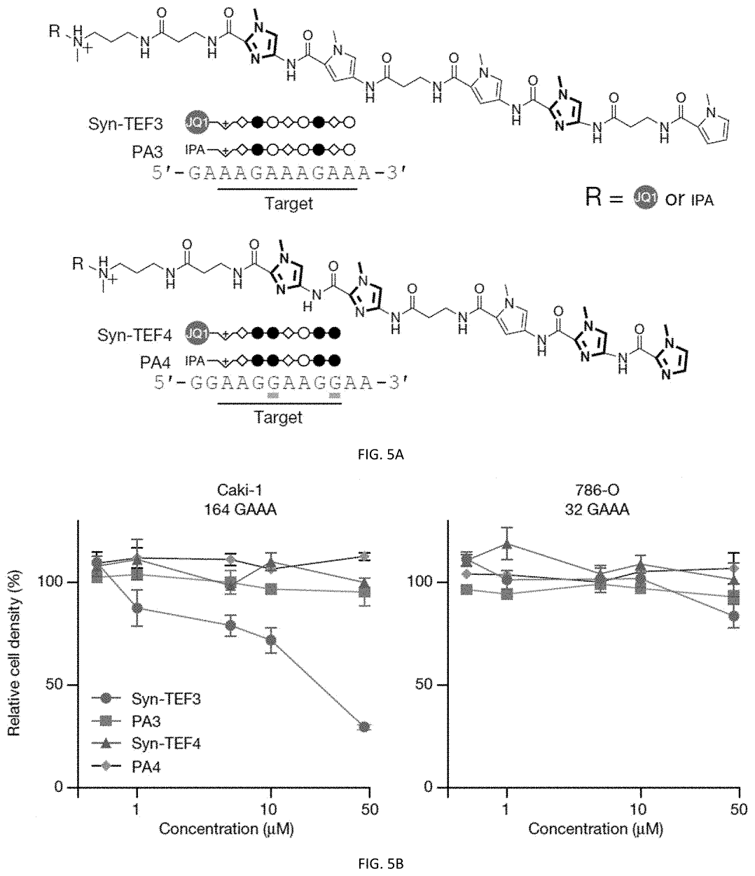

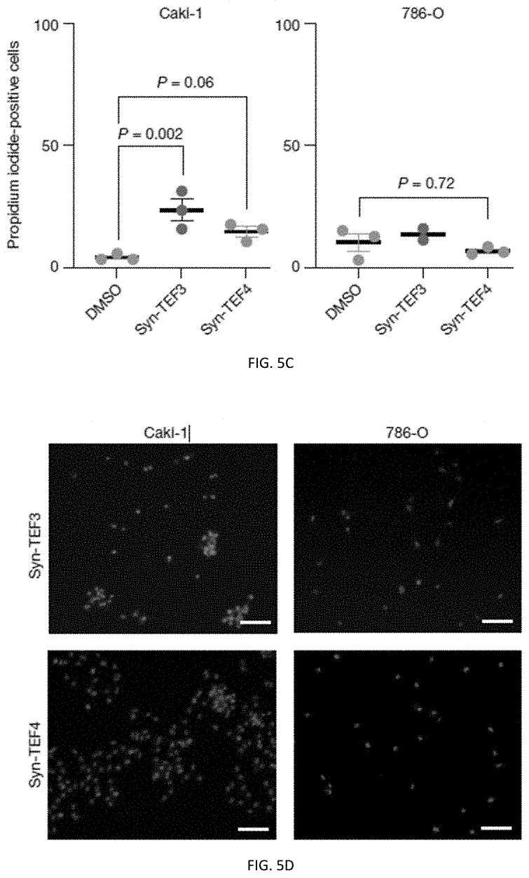

A synthetic transcription elongation factor, Syn-TEF3, was rationally designed to target GAAA and reverse gene misexpression in the vicinity (FIG. 5A). This molecule contains a GAAA-targeting polyamide (PA), and a bromodomain ligand, JQ1, designed to recruit the transcription elongation machinery. Alongside Syn-TEF3, a control molecule, Syn-TEF4, which targets GGAA TRs, as well as polyamides (PAs) PA3 and PA4 that lack the JQ1 domain were also designed.

The effect of Syn-TEFs on cell proliferation was next examined (FIG. 5D). Caki-1 and 786-o were selected because they have the largest (˜164) and smallest (˜32) GAAA tracts within the first intron of UGT2B7 , respectively. In a dose-dependent manner, Syn-TEF3 led to a significant decrease in the proliferation of Caki-1 cells but had negligible effect on 786-o cells. Syn-TEF4, which does not target a GAAA TR, did not significantly decrease proliferation in either of the cell lines tested, demonstrating the requirement for GAAA-specific DNA targeting.

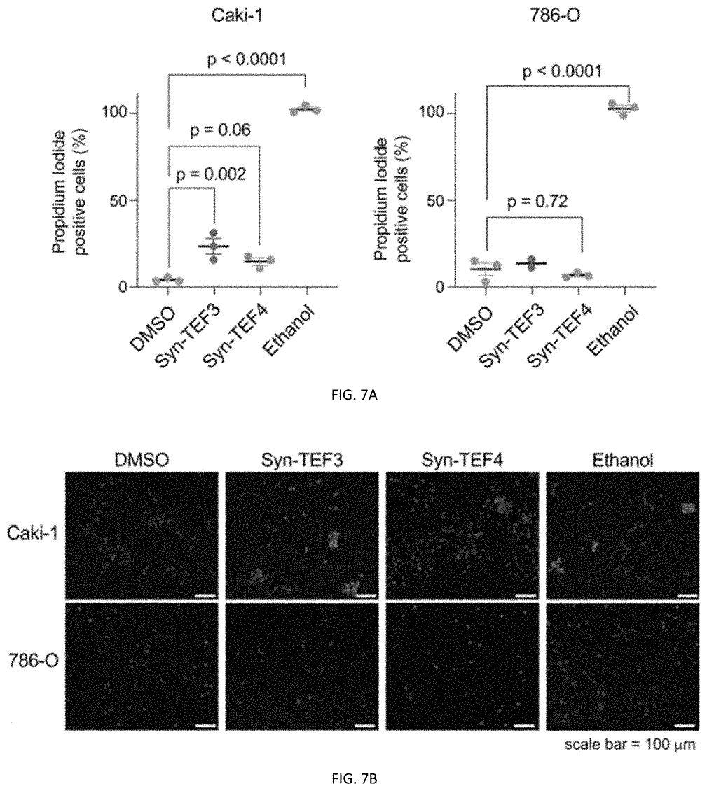

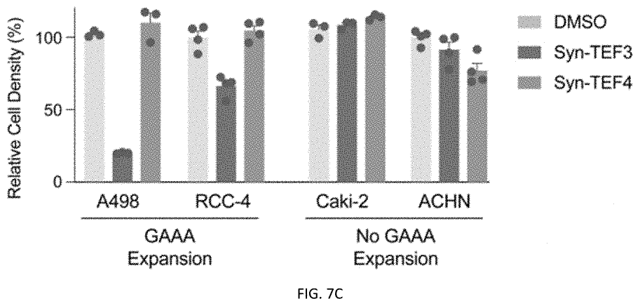

Two additional cell lines with GAAA-repeat expansions as well as two additional control non-expanded cell lines showed a similar association between Syn-TEF sensitivity and presence of the repeat expansion (FIGS. 7A-7C). In line with this finding, Caki-1 cells treated with Syn-TEF3 exhibited a significant increase in cell death when compared with the DMSO-treated control, as measured by propidium iodide staining (FIGS. 5C, 5D and 7A-7C). By contrast, 786-O cells treated with Syn-TEF3 showed no significant difference in propidium iodide-positive cells when compared with DMSO-treated cells (FIGS. 5C, 5D and 7A-7C). Notably, the Syn-TEF4, PA3 and PA4 control agents had no significant effect on cell death in either cell line when compared with vehicle control (FIGS. 5C, 5D and 7A-7C).

### Materials and Methods

Data curation White-listed data was obtained from the International Cancer Genome Consortium (ICGC) and The Cancer Genome Atlas (TCGA) pan-cancer analysis of whole genomes (PCAWG) dataset. Data were accessed through the Cancer Genome Collaboratory. The aligned reads (bam files) were used, which were aligned to GRCh37. These data are available through the PCAWG data portal.

Identification of somatic recurrent repeat expansions Tumor and matching normal samples were independent analyzed for each cancer type. ExpansionHunter De Novo (EHdn) (v0.9.0) was implemented with the following parameters: —min-anchor-mapq 50—max-irr-mapq 40. To prioritize loci, workflow termed Tandem Repeat Locus Prioritization in Cancer (TROPIC) was developed. Loci were included from chrl-22, X, and Y for downstream analysis. Loci were removed where >10% of Anchored in-repeat read (IRR) values were >40, which is the theoretical maximum value. The p-value (a non-parametric one-sided Wilcoxon rank sum test) for each locus was used to calculate a false discovery rate (FDR) q-value. Loci with FDR <0.10 are reported. Loci were selected where >5% of samples had an Anchored IRR Quotient >2.5. For a repeat expansion to be detected by ExpansionHunter De Novo, the tandem repeat was larger than the sequencing read length. A somatic repeat expansion was defined as having an FDR q-value <0.05 between tumor and normal samples. To call repeat expansions in individual cancer samples, the distribution of tumor and normal Anchored IRR values was analyzed and a conservative threshold for the Anchored IRR Quotient ((Tumor Anchored IRR—Normal Anchored IRR)/(Normal Anchored IRR+1))>2.5) was selected.

Local read depth normalization EHdn normalized the number of Anchored IRRs for a given locus to the global read depth. To account for chromosomal amplifications and other forms of genetic variation that can alter local read depth, the following normalization was performed. For each rRE locus and sample in its corresponding cancer, samtools v1.13 was used with the parameter depth -r to find the read depth at each base pair within the locus and a 500 bp region surrounding the start and stop positions of the TR. Next, the average read depth was calculated at each base pair and define this as the local read depth. Finally, the local read depth-normalized Anchored IRR value specific to a sample and rRE combination was calculated by dividing the Anchored IRR value from EHdn by the local read depth at the locus.

Generation of CABOSEN cell line CABOSEN cells were generated from a cabozantinib-sensitive (CABOSEN) human papillary RCC xenograft tumor grown in RAG2−/− gammaC−/− mice, as described previously (Zhao, H., et al., Cancer Biol. Ther. 18, 863-871 (2017)). Tumor tissue was minced with a sterile blade and the cell suspension cultured in DMEM/F-12 medium (Corning) supplemented with 10% (v/v) Cosmic Calf Serum (ThermoFisher). Cells were expanded and cryopreserved in growth medium supplemented with 10% (v/v) DMSO and cells from passage 8 were used for analysis.

Analysis of rREs by gel electrophoresis PCR was performed with CloneAmp HiFi PCR Mix (Takara Biosciences, Mountain View, CA) and added DMSO to a final concentration of 5-10% as needed. All cell lines were tested negative for mycoplasma contamination with the MycoAlert Mycoplasma Detection Kit (Lonza). Cell line identities were authenticated by STR profiling by the Genetic Resources Core Facility at Johns Hopkins University, with the exception of SNU-349, which did not match the reported STR profile of SNU-349 or any other catalogued cell line, but has a mutated VHL gene and expresses high levels of PAX8 and CA9, consistent with ccRCC origin.

Visualization of repeat expansions with ExpansionHunter and REViewer To inspect the reads supporting a repeat expansion, the repeat as described on the GitHub page for ExpansionHunter was annotated. The region with ExpansionHunter (v4.0.2) was then profiled using the default settings. The resulting reads were visualized with REViewer (v0.1.1) using the default settings. REViewer is available at github.com/Illumina/REViewer. A repeat expansion was called when the repeat tract length for one allele of the tumor sample was greater than 100 bp and exceeded the repeat tract length of either normal allele. A locus was called validated if at least 10 cancer genomes had a repeat expansion.

Validation of rREs in independent cohorts of samples Twelve pairs of matching normal and tumor samples from patients with clear cell renal cell carcinoma were obtained with the patients' informed consent ex vivo upon surgical tumor resection (Stanford IRB-approved protocols #26213 and #12597) and analyzed. Eighteen pairs of matching normal and prostate tumor samples were obtained from the Tissue Procurement Shared Resource facility at the Stanford Cancer Institute and analyzed. Fifteen pairs of matching normal and breast tumor samples were obtained from the Tissue Procurement Shared Resource facility at the Stanford Cancer Institute and analyzed. Nucleic acid was isolated with either the Quick Microprep Plus kit (Catalog D7005) or the Zymo Quick Miniprep Plus kit (Catalog D7003) (Zymo Research, Irvine, CA). Gel electrophoresis was performed as described above. A locus was called detected if a somatic repeat expansion was identified in at least one patient tumor sample compared to matching normal.

Downsampling analysis For the downsampling analysis, tumour genomes from RCC samples were downsampled from their mean (52x) sequencing depth to 40×, 30×, 20× and 10× depth with the samtools view command. EHdn was run, as described above, for each of the sequencing depths, and the Bonferroni-corrected P value was plotted for the rRE in UGT2B7 (GAAA, chr4:69929297-69930148).

Benchmarking the local read depth normalization filter The local read depth filter was benchmarked in silico by observing its behavior with simulated reads. First, a reference genome containing artificially expanded repeats was created. Ten TRs located on chromosome 1 that were shorter than the sequencing read length of 100 bp were randomly selected. These TRs were artificially expanded on chromosome 1 of GRCh37 with the BioPython Python package (v1.79). Next, wgsim (v0.3.1-r13) was used to simulate reads from the reference file with the command ‘wgsim -N 291269925—1 100—2 100 reference file. fasta output.read1.fastq output.read2.fastq’. The number of reads (specified by the -N option) was calculated to achieve 30× coverage of chromosome 1. The resulting pair of files, hereafter referred to as the base fastq files, contained a copy number of 2 for all of the expansions.

To simulate copy number amplification, the read simulation process was repeated using reference files that contained only the artificially expanded repeats and their surrounding 1,000-bp flanking regions. Ten pairs of fastq files were created, each with an increasing copy number. The copy number was specified by multiplying the number of reads to generate (wgsim -N option) by the required number. To generate the final set of fastq files, each pair of copy number-amplified fastq files was concatenated with the base fastq files. The end result was eight pairs of fastq files that contained reads for chromosome 1 and copy number amplification varying from 2 to 10 of the expanded repeats.

The base fastq file with a copy number of 2, in addition to the eight copy number-amplified fastq files, was aligned to chromosome 1 of GRCh37 with bwa-mem (v0.6) with the default options. The resulting SAM files were converted to BAM format with samtools (v1.15) using the default options. Finally, the EHdn profile command (v0.9.0) was run with the minimum anchor mapping quality set to 50 and maximum IRR mapping quality set to 40. Finally, the Anchored IRR values were extracted by overlapping the STR coordinates with the de novo repeat expansion calls.

Short-read and long-read DNA sequencing The Caki-1 and 786-O cell lines were sequenced with both short-read sequencing (60× sequencing coverage, 150-bp paired-end sequencing on a NovaSeq 6000 instrument) and long-read sequencing (50× sequencing coverage, PacBio HiFi sequencing on a Sequel IIe instrument). The long reads were aligned to GRCh37 with pbmm2 (v1.7.0), using the parameters—sort—min-concordance-perc 70.0—min-length 50. The short reads were aligned to GRCh37 with Sentieon (v202112.01) using parameters -K 10000000 -M, an implementation of BWA-MEM, and analysed the samples with EHdn, as described above. Loci were included for which at least one sample had an Anchored IRR value of >0 for further analysis. Anchored IRR values >0 arise when the repeat length exceeds the sequencing read length. To benchmark EHdn against long-read sequencing data, the TR length of a given locus was manually determined in the long-read sequencing data. If the TR length in the long-read sequencing data exceeded the short-read sequencing read length of 150 bp, that locus was considered to have been confirmed.

The PacBio HiFi data were aligned to GRCh37 with pbmm2 (v1.7.0) and visualized at the UGT2B 7 locus with Tandem Repeat Genotyper (v0.2.0; github.com/PacificBiosciences/trgt).

Analysis of rRE loci To determine if rREs were associated with any human diseases, rREs were mapped to genes with GREAT (v4.0.4, default settings). The resulting genes were analyzed with Enrichr using Jansen Diseases. To determine whether repeat expansions were associated with microsatellite instability-high (MSI-High) cancers, data was obtained from Hause et al. (Nat. Med. 22, 1342-1350 (2016)). The percentage of MSI-high cancers were obtained from colon adenocarcinoma (COAD), stomach adenocarcinoma (STAD), kidney renal cell carcinoma (KIRC), ovarian serous cystadenocarcinoma (OV), prostate adenocarcinoma (PRAD), head and neck squamous cell carcinoma (HNSC), liver hepatocellular carcinoma (LIHC), bladder urothelial carcinoma (BLCA), glioblastoma multiforme (GBM), skin cutaneous melanoma (SKCM), thyroid carcinoma (THCA), and breast invasive carcinoma (BRCA) and compared to the number of repeat expansions and the percentage of patients with at least one repeat expansion in the corresponding cancer type from the PCAWG dataset. Cancer genomes containing rREs were overlapped with the microsatellite mutation rate, termed the STR mutation rate, and MSI calls from Fujimoto et al. (Genome Res. 30, 334-346 (2020)). Association of rREs with STR mutation rate was assessed with the two-tailed Wilcoxon rank sum test. Association of rREs with MSI calls was assessed with Chi-square test with Yates' correction.

To determine whether rREs are associated with known mutational signatures, mutational signatures were downloaded from the ICGC DCC

(dcc.icgc.org/releases/PCAWG/mutational_signatures/Signatures_in_Samples). A multiple linear regression was performed for each single-base-substitution (SBS) and doublet-base-substitution (DB S) signatures to identify predictors of the number of rREs present in a sample. To choose the predictors best subset selection was performed on DBS and SBS signatures and age was included as a possible confounding factor. Statsmodels v0.12.2 in Python and, specifically, the ordinary least squares model found in the statsmodels.api.OLS module was used to estimate the coefficients of the selected predictors in their corresponding multiple linear regression model.

To determine whether repeat expansions were associated with a difference in cytotoxic activity, cytotoxic activity was calculated as previously described for four cancers that had matching RNA-seq and WGS (Rooney, M. S., et al., Cell 160, 48-61 (2015)). For each locus, cytolytic activity for patients with a repeat expansion were compared to patients without a detected repeat expansion using a Welch's t-test with correction for multiple hypothesis testing (Benjamini-Hochberg FDR q-value <0.05). rREs were annotated with genic elements using Annotatr (v1.18.1).

To determine if rREs were associated with regulatory elements, candidate cis-regulatory elements (cCREs) were downloaded and mapped to GRCh37 with liftover (UCSC). The distance between rREs and cCREs was determined with bedtools closest command (v2.27.1) and compared to the simple repeats catalog. To compare the distance to ENCODE cCREs, a Welch's t-test was performed.

To determine if prostate cancer rREs were associated with prostate cancer susceptibility loci, the distance to three sets of loci was calculated using the “bedtools closest” command. The distances between (1) rREs present in prostate cancer samples and prostate cancer susceptibility loci, (2) rREs not present in cancer samples and cancer susceptibility loci, and (3) simple repeats and cancer susceptibility loci were calculated. To compare the distances between these three associations, a Welch's t-test with FDR correction (Benjamini-Hochberg) was performed.

To determine whether rREs were associated with replication timing, Repli-seq replication timing data was downloaded from seven cell lines from the ENCODE website (NCI-H460, T470, A549, Caki2, G401, LNCaP, and SKNMC). Regions for which all cell lines had concordant signals were selected for analysis (early or late replication designations agreed for each cell line at a given locus). Whether there was a difference in the distribution of rREs across early- and late-replicating regions compared to the simple repeats catalog with a bootstrapping (n=10,000) was determined. 54 loci (the number of rREs that are present in a concordant replication region) were sampled from rREs and simple repeats. A Welch's t-test was performed on the bootstrapped samples to estimate a p-value. FDR correction (Benjamini-Hochberg) was applied to the estimated p-values. To determine whether rRE status in UGT2B7 was associated with survival outcome in clear cell RCC patients (TCGA abbreviation: KIRC), Welch's t-test quartile was used.

To identify motifs enriched and depleted in the rRE catalogue, the same method as in the motifscan Python module (v1.3.0) was followed. The rRE catalogue was compared to the simple repeats catalogue (TRF) as a control. For each unique motif present, a contingency table specifying the count of rREs and simple repeats with and without the motif was built. Two one-tailed Fisher's exact tests were applied to the table to test for significance in both directions, that is, enrichment and depletion. The ‘stats’ module in the Scipy Python package (v1.7.0) was used to conduct the significance test. Because multiple-hypothesis tests were performed, FDR correction (Benjamini—Hochberg) was applied for multiple-hypothesis testing to the P values, with a cut-off (FDR) of 0.01.

For the comparison of SNVs in COSMIC genes to rREs, the cancer genomes were first divided into two categories: an rRE cohort and a non-rREcohort. The rRE cohort contained all genomes that had at least one rRE detected (n=615), and the non-rRE cohort contained all genomes that had no rREs detected (n=1,897). The number of donors in the rRE cohort that had at least one mutation in a given gene (COSMIC tier 1 genes) i and the number of donors in the non-rRE cohort that had at least one mutation in a given gene i with a contingency table were then looked at. The P value (Fisher's exact test) was calculated for the significance of associating genes with either the rRE or non-rRE cohort. This P-value calculation was repeated for all COSMIC genes, using FDR at a significance level of 0.05 (Benjamini—Hochberg) to correct for multiple-hypothesis testing.

Estimation of expansions in the general population To estimate the frequency of rREs in the general population, EHdn (v0.9.0) was run on 1000 Genomes Project samp1es60 (n=2,504) (GRCh38) and Medical Genome Reference Bank61 samples (n=4,010) (GRCh37 lifted over to GRCh38).

The genomic coordinates of the 160 rREs (GRCh37) were padded with 1,000 bp and translated to GRCh38 coordinates with UCSC LiftOver. Then, the rRE coordinates (GRCh38) were overlapped with loci from the population samples containing Anchored IRR calls. rREs that overlapped with matching motifs in the population samples were selected for further analysis. To identify expanded rREs in the population samples and to quantify their prevalence, their global-normalized Anchored IRR values were converted to be comparable to ICGC values. This step was utilized because sequencing read lengths in the PCAWG dataset are generally 100 bp while the read lengths in the 1000 Genomes and Medical Genome Reference Bank datasets are 150 bp. Conversion followed the formula (Anchored IRR, 100 bp)=0.5+1.5×(Anchored IRR, 150 bp). A sample in the population samples was counted as expanded if its Anchored IRR value was greater than the 99th percentile of Anchored IRR values in the normal samples from the PCAWG dataset, a threshold that is comparable to the threshold used to call expansions in tumour samples. In future rRE catalogues, for the rare instance where the estimated frequency of repeat expansions in the population samples is higher than expected, these data could be used to further filter rREs to improve the detection of cancer-specific repeat expansions.

To compare the length of TRs in normal samples with and without a matching rRE in a tumour sample, donors in the Prost-AdenoCA and Kidney-RCC cohorts whose data are available for download through the Cancer Collaboratory were included (n=253). ExpansionHunter (v5.0.0) was used with the default options to genotype prostate and kidney cancer rREs in the normal samples of the selected donors. When there were two alleles of an rRE in a sample, both alleles were included and treated as distinct data points. For each rRE, the distribution of genotypes from donors who had an expansion in their tumor samples was tested to determine whether it differed from that for donors who did not have an expansion. Student's t test was used to compute P values with FDR correction (Benjamini-Hochberg) to adjust for multiple-hypothesis testing.

Association of rREs with gene expression Matching RNA-seq and WGS data were available for Kidney-RCC, Ovary-AdenoCA, Panc-AdenoCA, and Panc-Endocrine. RNA-seq data from these samples were obtained from DCC and values were converted to transcripts per million (TPM). Normalized gene expression (TPM) values were compared for samples with and without an rRE (Welch's t-test, with FDR correction. For isoform analysis, normalized gene expression counts were compared for samples with and without a repeat expansion using the DESeq2 (v1.32.0) package in R v4.0.5. The DESeq function was used to calculate the loge fold change values for 3 isoforms of the UGT2B7 gene (ENST00000305231.7, ENST00000508661.1, ENST00000502942.1) and performed a Wald test with FDR correction using the Benjamini-Hochberg procedure (threshold q-value <0.01).

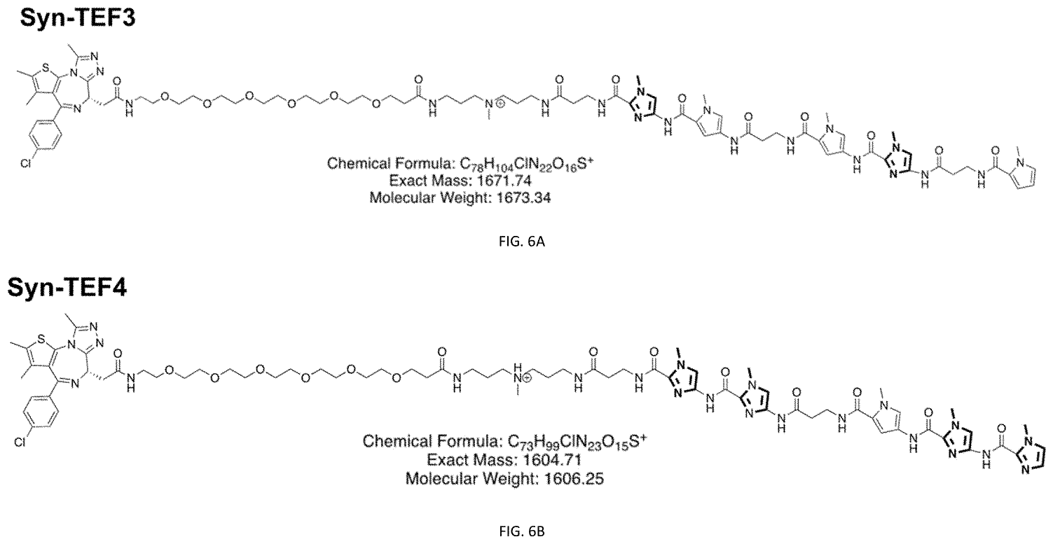

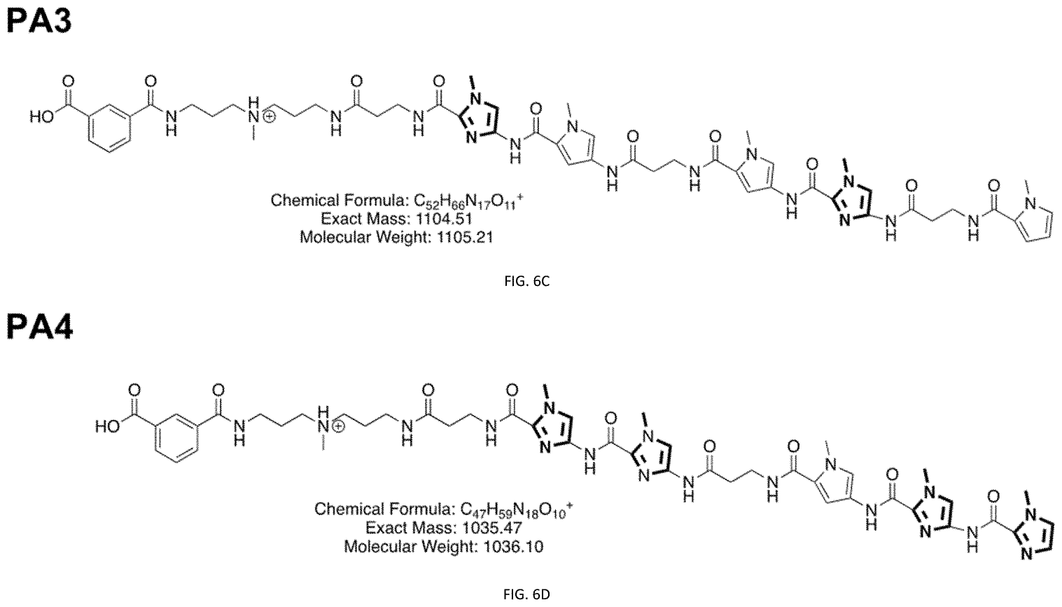

Design, synthesis, and characterization of Syn-TEFs and Pas Synthetic transcription elongation factors (Syn-TEFs) and polyamides (PAs) were designed to target a GAAA repeat (Syn-TEF3 and PA3) or a control GGAA repeat (Syn-TEF4 and PA4). Syn-TEF3, Syn-TEF4, PA3, and PA4 were synthesized and purified to a minimum of 95% compound purity by WuXi Apptec and used without further characterization. HPLC conditions for chemical characterization: 1.0 mL/min, Solvent A: 0.1% trifluoroacetic acid (TFA) in H2O, Solvent B: 0.075% TFA in acetonitrile, Gemini, Column: C18 5 mm 110A 150*4.6 mm. Full results of characterization can be found in FIG. 6.

Treatment of RCC cell lines with synthetic transcription elongation factors (Syn-TEFs) Caki-1, Caki-2, and 786-o cells were obtained from ATCC and grown in RPMI 1640 media with L-glutamine (Gibco Catalog 11875093), supplemented with 10% FBS. A498 and ACHN cells were obtained from ATCC and grown in DMEM with glucose, 1-glutamine and sodium pyruvate (Corning, 10-013-CV), supplemented with 10% (vol/vol) FBS. RCC-4 cells were obtained from A. Giacca (Stanford University) and grown in DMEM with glucose, 1-glutamine and sodium pyruvate (Corning, 10-013-CV), supplemented with 10% (vol/vol) FBS.Cell lines were confirmed by STR profiling (Genetic Resource Core Facility, Johns Hopkins University) and tested negative for mycoplasma. Cells were seeded in 96-well plates on Day 0. On Day 1, cells were treated with the indicated molecules. Molecules were dissolved in DMSO (vehicle) and added to cells (0.1% DMSO final concentration). On Day 4 (72 h later), relative metabolic activity was used as a proxy for relative cell density, was measured with the Cell Counting Kit (CCK-8; Dojindo Molecular Technologies) per the manufacturer's instructions. Absorbance (450 nm) of cells treated with molecules were normalized to DMSO (0.1%) or no treatment. Absorbance was measured with an Infinite M1000 microplate reader (Tecan, Mannedorf, Switzerland).

For microscopy, Caki-1 and 786-O cells were plated on glass-bottom 96-well plates under standard culture conditions. One day after plating, medium containing no drug, 50 μM Syn-TEF3 or 50 μM Syn-TEF4 was added, and the cells were incubated for 72 h at 37° C. As a control, wells that received no treatment were incubated with 70% (vol/vol) ethanol for 30 s before staining. Cells were then stained with propidium iodide, Calcein-AM and Hoechst 33342 from the Live-Dead Cell Viability Assay kit (Millipore Sigma, CBA415) according to the manufacturer's instructions and immediately imaged at ×10 magnification with a 0.17-NA CFI60 objective on a Keyence BZ-X710 microscope. Eight fields were measured for each treatment condition, and the experiment was repeated two times. Quantification was conducted using FIJI software (release 20220330-1517). For statistical analyses, one-way ANOVA adjusted with Bonferroni correction for multiple comparisons was conducted with GraphPad Prism (v9.3.1).

Statistics and reproducibility Data are represented as the mean±s.e.m. unless stated otherwise. All experiments were reproduced at least twice unless stated otherwise. Box plots were prepared with matplotlib (v3.4 or v3.6) as follows unless stated otherwise: the box extends from the first quartile (Q1 or 25^{th} percentile) to the third quartile (Q3 or 75th percentile) of the data, with a line at the median. The whiskers extend from the box by 1.5 times the interquartile range (IQR). The IQR is the difference between the values at Q3 and Q1. Outliers were not plotted to improve clarity. Details on how box plots were generated are available at matplotlib.org/stable/api/_as_gen/matplotlib.axes.Axes.boxplot.html#matplotlib.axes.Axes.boxpl ot.

TABLE 2

| chromosome | start | stop | Motif | SEQ ID NO | Cancer |
|---|---|---|---|---|---|
| chr1 | 2052209 | 2053620 | AACCACCACCGT | 1 | Prostate-AdenoCA |
| | | | GACCCT | | |
| chr1 | 4195391 | 4197375 | AACCCACTCCCAT | 2 | Prostate-AdenoCA |
| | | | GATAACT | | |
| chr1 | 5855301 | 5856988 | ACCACCAGGGCT | 3 | Prostate-AdenoCA |
| | | | CAGTC | | |
| chr1 | 19251719 | 19252987 | ATCC | — | Prostate-AdenoCA |
| chr1 | 41996161 | 41998397 | ACAGGAGAGATG | 4 | Prostate-AdenoCA |
| | | | GAGG | | |
| chr1 | 57222577 | 57224042 | AAAG | — | Liver-HCC |
| chr1 | 80399362 | 80400680 | AAAG | — | Prostate-AdenoCA |
| chr1 | 84266630 | 84268313 | AAAG | — | Liver-HCC |
| chr1 | 100147590 | 100149506 | AAAG | — | Prostate-AdenoCA |
| chr1 | 100147638 | 100150409 | AAAG | — | Liver-HCC |
| chr1 | 100147754 | 100149508 | AAAG | — | Ovary-AdenoCA |
| chr1 | 152205843 | 152207283 | AACTATATATAT | 5 | Liver-HCC |
| chr1 | 155268413 | 155269183 | AAAG | — | Kidney-RCC |
| chr1 h | 162018882 | 162020227 | AAAG | — | Prostate-AdenoCA |
| chr1 | 166078198 | 166079854 | AT | — | Liver-HCC |
| chr1 h | 213536054 | 213538012 | AAAG | — | Liver-HCC |
| chr1 | 235130783 | 235132289 | ACATATATACCTA | 6 | Liver-HCC |
| | | | TATAT | | |
| chr1 | 248326821 | 248329129 | ACTGGAGCCCCCT | 7 | Prostate-AdenoCA |
| | | | GAGG | | |
| chr10 | 3260228 | 3262140 | ATCC | — | Prostate-AdenoCA |
| chr10 | 10307335 | 10309851 | AACACCAGCGTC | 8 | Prostate-AdenoCA |
| | | | ATG | | |
| chr10 | 10450565 | 10452069 | ACAGAGCTGATC | 9 | Prostate-AdenoCA |
| | | | CATGCCCC | | |
| chr10 | 32403273 | 32405038 | AAG | — | Prostate-AdenoCA |
| chr10 | 47588837 | 47590728 | ACCATCCTCAGCT | 10 | Prostate-AdenoCA |
| | | | CACTCC | | |
| chr10 | 73179499 | 73180744 | ACCATCATCATC | 11 | Prostate-AdenoCA |
| chr10 | 100035160 | 100036606 | AAG | — | Prostate-AdenoCA |
| chr11 | 1795780 | 1798189 | AGAGGGGATGG | 12 | Prostate-AdenoCA |
| chr11 | 1842240 | 1843521 | ATCC | — | Prostate-AdenoCA |
| chr11 | 36075829 | 36076848 | ATCC | — | Prostate-AdenoCA |
| chr11 | 61440544 | 61442229 | ATCC | — | Prostate-AdenoCA |
| chr11 | 68783270 | 68785209 | ATCC | — | Prostate-AdenoCA |
| chr11 | 68877246 | 68879063 | ATCC | — | Prostate-AdenoCA |
| chr11 | 69029824 | 69031322 | ATCC | — | Prostate-AdenoCA |
| chr11 | 69686131 | 69687976 | ACCCATCACGCCC | 13 | Prostate-AdenoCA |
| | | | ACCTGG | | |
| chr11 | 71113533 | 71115810 | AAGGAGATGGAG | 14 | Prostate-AdenoCA |
| | | | GCTCAGAG | | |
| chr11 | 83810142 | 83811719 | AAAGAGATATAT | 15 | Liver-HCC |
| | | | ATATCT | | |
| chr12 | 96047849 | 96049813 | ATCATCCC | — | Prostate-AdenoCA |
| chr12 | 104046690 | 104048212 | ATCC | — | Prostate-AdenoCA |
| chr12 | 108349869 | 108351628 | AAG | — | Prostate-AdenoCA |
| chr12 | 126010680 | 126012701 | AAGGGTGGATGG | 16 | Prostate-AdenoCA |
| | | | GTGGATGG | | |
| chr12 | 127315830 | 127317099 | AGG | — | Prostate-AdenoCA |
| chr12 | 131520424 | 131522359 | ACCGGGCCTCACT | 17 | Prostate-AdenoCA |
| | | | CACTGC | | |
| chr13 | 113983725 | 113986581 | ACACACCTGGGC | 18 | Prostate-AdenoCA |
| | | | TCCCATGC | | |
| chr13 | 114124505 | 114126087 | AACTCCACAGAG | 19 | Prostate-AdenoCA |
| | | | GACCC | | |
| chr14 | 25165002 | 25166658 | AAGGTGAGTGAG | 20 | Prostate-AdenoCA |
| | | | TGG | | |
| chr14 | 89262766 | 89263437 | AACATATAATAT | 21 | CNS-PiloAstro |
| | | | ATAT | | |
| chr14 | 101710579 | 101713704 | AAACCTCGCATCC | 22 | Prostate-AdenoCA |
| | | | TATCT | | |
| chr14 | 104669272 | 104671705 | ATCC | — | Prostate-AdenoCA |
| chr14 | 104764651 | 104766341 | ATCC | — | Prostate-AdenoCA |
| chr14 | 106048515 | 106049602 | ACCAGGGCTCAG | 23 | Prostate-AdenoCA |
| | | | TGATCAGG | | |
| chr15 | 30241059 | 30242892 | ACC | — | Prostate-AdenoCA |
| chr15 | 69592301 | 69593910 | AAAG | — | Prostate-AdenoCA |
| chr16 | 1082473 | 1084957 | ATCC | — | Prostate-AdenoCA |
| chr16 | 25884115 | 25885402 | ATCC | — | Prostate-AdenoCA |
| chr16 | 86922061 | 86923838 | AATGGATGGATG | 24 | Prostate-AdenoCA |
| | | | GATGGATG | | |
| chr17 | 3422199 | 3423724 | ATCC | — | Prostate-AdenoCA |
| chr17 | 26842230 | 26845631 | ACACACCTCCAC | 25 | Prostate-AdenoCA |
| | | | AGGGT | | |
| chr17 | 40490521 | 40491274 | AAGGAGTATTCC | 26 | Prostate-AdenoCA |
| | | | CTCAGGTC | | |
| chr17 | 75211238 | 75213359 | AACCCTACCTACT | 27 | Prostate-AdenoCA |
| chr17 | 77866940 | 77868482 | ATC | — | Prostate-AdenoCA |
| chr17 | 78673700 | 78675010 | ACC | — | Prostate-AdenoCA |
| chr17 | 78716880 | 78719390 | ACAGCCCTGGCT | 28 | Prostate-AdenoCA |
| | | | ACTAGC | | |
| chr17 | 78903475 | 78904731 | AGCTCATCCCC | 29 | Prostate-AdenoCA |
| chr17 | 81065192 | 81066856 | AAGGATCGTGCA | 30 | Prostate-AdenoCA |
| | | | GCGAGG | | |
| chr18 | 19648051 | 19649681 | AAG | — | Prostate-AdenoCA |
| chr18 | 22518980 | 22519807 | AAATTTATATACA | 31 | CNS-PiloAstro |
| | | | TAT | | |
| chr18 | 23882552 | 23884210 | AAAGTGGAGGGA | 32 | Prostate-AdenoCA |
| | | | GGATG | | |
| chr18 | 62211903 | 62213253 | ACCCTATATATAT | 33 | Liver-HCC |
| chr18 | 64470723 | 64472391 | AATATAATATAAT | 34 | Liver-HCC |
| | | | ATATAT | | |
| chr18 | 75380431 | 75383735 | ACGCCAGTCTCTG | 35 | Prostate-AdenoCA |
| | | | CCCC | | |
| chr18 | 76252603 | 76254678 | ACCATCCCCCTCA | 36 | Prostate-AdenoCA |
| | | | GTGAGTC | | |
| chr18 | 77832358 | 77834330 | ACAGAGTCCCAG | 37 | Prostate-AdenoCA |
| | | | AGC | | |
| chr19 | 6775678 | 6777292 | ATCC | — | Prostate-AdenoCA |
| chr19 | 8787324 | 8789153 | ATCC | — | Prostate-AdenoCA |
| chr19 | 13705396 | 13707024 | ATCC | — | Prostate-AdenoCA |
| chr19 | 15888492 | 15890848 | ACTCACTCCCTCC | 38 | Prostate-AdenoCA |
| | | | CCTCCTC | | |
| chr19 | 33229818 | 33231921 | ACCCAGGAGATG | 39 | Prostate-AdenoCA |
| | | | CAGAGAGT | | |
| chr19 | 49684746 | 49686166 | ATCC | — | Prostate-AdenoCA |
| chr19 | 51001474 | 51003542 | AG | — | Prostate-AdenoCA |
| chr19 | 56360581 | 56363648 | AAATTACACCGC | 40 | Prostate-AdenoCA |
| | | | AGCCTCGG | | |
| chr2 | 9916302 | 9917404 | ACCCATCCATCCA | 41 | Prostate-AdenoCA |
| | | | TCC | | |
| chr2 | 11092782 | 11094781 | AACCACATCCAC | 42 | Prostate-AdenoCA |
| | | | ATCCAC | | |
| chr2 | 33141292 | 33141321 | ACCCCCCCCCCCC | 43 | Kidney-ChRCC |
| | | | CCCCCCC | | |
| chr2 | 95702030 | 95705646 | ACTC | — | Prostate-AdenoCA |
| chr2 | 123991498 | 123993020 | AGCATATATAT | 44 | Liver-HCC |
| chr20 | 20910677 | 20912389 | ACCATCCCCTCCC | 45 | Prostate-AdenoCA |
| | | | TATCCCC | | |
| chr20 | 43066391 | 43067071 | AAATATATAATAT | 46 | CNS-PiloAstro |
| | | | AT | | |
| chr20 | 62062720 | 6206471 | AAGGCCCCACCC | 47 | Prostate-AdenoCA |
| | | | TCAGGG | | |
| chr20 | 62076417 | 62078096 | ACAGGGCCCCCC | 48 | Prostate-AdenoCA |
| | | | AGCTCAG | | |
| chr21 | 10818232 | 10821091 | AATGCAATGGAA | 49 | Liver-HCC |
| | | | TGGAATGG | | |
| chr21 | 30931797 | 30933643 | AAAG | — | Prostate-AdenoCA |
| chr21 | 47784417 | 47786438 | AACAGAACCCCA | 50 | Prostate-AdenoCA |
| | | | CGGCAGTG | | |
| chr22 | 29262449 | 29263970 | AAAG | — | Liver-HCC |
| chr22 | 43973867 | 43976039 | AATGATGCCTGC | 51 | Prostate-AdenoCA |
| | | | ATGTAGGG | | |
| chr22 | 45475427 | 45477023 | ATC | — | Prostate-AdenoCA |
| chr3 | 3638741 | 3640142 | ACACACACACAT | 52 | Prostate-AdenoCA |
| | | | ATATAT | | |
| chr3 | 63299150 | 63300350 | ATATATATATATA | 53 | Liver-HCC |
| | | | TCC | | |
| chr3 | 152411463 | 152412776 | AATATATATATGG | 54 | Liver-HCC |
| chr3 | 181007995 | 181008723 | AT | — | Ovary-AdenoCA |
| chr4 | 7696475 | 7698260 | ATCC | — | Prostate-AdenoCA |
| chr4 | 34581896 | 34583228 | AAAG | — | Prostate-AdenoCA |
| chr4 | 49092873 | 49097336 | AATGG | — | Liver-HCC |
| chr4 | 69929297 | 69930148 | AAAG | — | Kidney-RCC |
| chr4 | 110770678 | 110772200 | AAAG | — | Liver-HCC |
| chr4 | 118987004 | 118987694 | AACATATAATAT | 55 | CNS-PiloAstro |
| | | | AT | | |
| chr4 | 186747859 | 186749338 | ACCATCATC | — | Prostate-AdenoCA |
| chr5 | 2584450 | 2586125 | ATCC | — | Prostate-AdenoCA |
| chr5 | 5815136 | 5816660 | ATATATATATATC | 56 | Liver-HCC |
| | | | C | | |
| chr5 | 5954129 | 5955785 | AAGGAGGGAAGG | 57 | Prostate-AdenoCA |
| | | | GG | | |
| chr5 | 49941722 | 49942207 | AAATATACAATT | 58 | CNS-PiloAstro |
| | | | ATATAT | | |
| chr5 | 50406264 | 50407898 | AAAG | — | Liver-HCC |
| chr5 | 77725192 | 77726133 | AAACAAATACTG | 59 | Liver-HCC |
| | | | TATTTAT | | |
| chr5 | 113953520 | 113955031 | AT | — | Liver-HCC |
| chr5 | 133270973 | 133272746 | AACCAGGGGAGG | 60 | Prostate-AdenoCA |
| | | | AGCCACAG | | |
| chr5 | 147081377 | 147083045 | AAAG | — | Liver-HCC |
| chr5 | 170180445 | 170182522 | ATCC | — | Prostate-AdenoCA |
| chr6 | 40175763 | 40178239 | ACATATATACATA | 61 | Liver-HCC |
| | | | TATGT | | |
| chr6 | 46711056 | 46712978 | AAG | — | Liver-HCC |
| chr6 | 49668523 | 49669988 | AATGTATACATAT | 62 | Liver-HCC |
| | | | AT | | |
| chr6 | 66726881 | 66728056 | ATATATATATATC | 63 | Liver-HCC |
| | | | C | | |
| chr6 | 67683909 | 67685017 | ATATATATGATAT | 64 | Liver-HCC |
| | | | ATC | | |
| chr6 | 92180554 | 92181567 | AATACATATATA | 65 | CNS-PiloAstro |
| | | | ATATAT | | |
| chr6 | 164891228 | 164892692 | AAGG | — | Prostate-AdenoCA |
| chr6 | 170483352 | 170487943 | AACAACCCCGAA | 66 | Prostate-AdenoCA |
| | | | CAGACAGT | | |
| chr7 | 282482 | 284248 | ACAGGTCTCCTGG | 67 | Prostate-AdenoCA |
| | | | GTGGC | | |
| chr7 | 37886176 | 37888865 | AAG | — | Prostate-AdenoCA |
| chr7 | 44346088 | 44347503 | ACACCCTCCTCCC | 68 | Prostate-AdenoCA |
| | | | CCTGCTC | | |
| chr7 | 45649852 | 45651024 | ACCATCACCTCCC | 69 | Prostate-AdenoCA |
| | | | CACCTCC | | |
| chr7 | 93226100 | 93227489 | AG | — | Liver-HCC |
| chr7 | 121243013 | 121243813 | AAAG | — | Lung-SCC |
| chr7 | 126542778 | 126544455 | ACACATATACAT | 70 | Liver-HCC |
| | | | ATATATAT | | |
| chr7 | 156309532 | 156311311 | ACACAGCCTCCCT | 71 | Prostate-AdenoCA |
| | | | C | | |
| chr7 | 157845527 | 157847271 | ACCCCAGAGATG | 72 | Prostate-AdenoCA |
| | | | CAGAG | | |
| chr7 | 158440671 | 158442225 | AAATGGACTATA | 73 | Prostate-AdenoCA |
| | | | ACCACGCC | | |
| chr8 | 41187308 | 41188296 | ATCC | — | Prostate-AdenoCA |
| chr8 | 57239613 | 57240997 | AAATATATATAT | 74 | Liver-HCC |
| chr8 | 73473300 | 73474607 | AT | — | Liver-HCC |
| chr8 | 77091835 | 77093099 | ACATATATACATA | 75 | Liver-HCC |
| | | | TATAT | | |
| chr8 | 82754050 | 82755313 | AG | — | Prostate-AdenoCA |
| chr8 | 101810990 | 101812809 | AAAG | — | Liver-HCC |
| chr8 | 109109164 | 109110910 | AAAG | — | Liver-HCC |
| chr8 | 121893676 | 121895308 | AAAG | — | Liver-HCC |
| chr8 | 132743064 | 132745145 | AAAG | — | Liver-HCC |
| chr8 | 143036455 | 143037824 | ACACATACATAT | 76 | Liver-HCC |
| | | | ATAT | | |
| chr8 | 143063399 | 143066586 | ACACTGACCATCC | 77 | Prostate-AdenoCA |
| | | | ATAGTCC | | |
| chr8 | 143336183 | 143338087 | ACCACCACCACC | 78 | Prostate-AdenoCA |
| | | | ACCATC | | |
| chr9 | 7193914 | 7195803 | ATCC | — | Prostate-AdenoCA |
| chr9 | 28188715 | 28190581 | AAGGAAGGAAGG | 79 | Prostate-AdenoCA |
| | | | AAGGGAGG | | |
| chr9 | 36869138 | 36870869 | ATCC | — | Prostate-AdenoCA |
| chr9 | 74034898 | 74036374 | AAAGAGAGAG | 80 | Liver-HCC |
| chr9 | 91291620 | 91292961 | ACAGTGAGGACC | 81 | Prostate-AdenoCA |
| | | | ATGGGG | | |
| chr9 | 138087381 | 138088897 | AAATGGGAAGGG | 82 | Prostate-AdenoCA |
| | | | G | | |
| chrX | 1800051 | 1801323 | AG | — | Prostate-AdenoCA |
| chrX | 2130341 | 2133043 | AAGGAAGGGAAG | 83 | Prostate-AdenoCA |
| | | | GAGG | | |
| chrX | 2221218 | 2222934 | ATCC | — | Prostate-AdenoCA |
| chrX | 4039079 | 4040587 | AAAG | — | Prostate-AdenoCA |
| chrX | 68032465 | 68033958 | AAAAATATATAT | 84 | Liver-HCC |
| chrX | 125168784 | 125170222 | AAATATATATAC | 85 | Liver-HCC |
| | | | ATATAT | | |
| chrX | 127660583 | 127661711 | AATATAGACTAT | 86 | Liver-HCC |
| | | | ATTATAT | | |
| chrX | 127660813 | 127661371 | AATATAGACTAT | 87 | CNS-PiloAstro |
| | | | ATTATAT | | |
| chrX | 138585430 | 138586380 | ACATATACGGAT | 88 | Liver-HCC |
| | | | AT | | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound, or a pharmaceutically acceptable salt thereof, having the structure:
A-B-C
wherein:
A is a bromodomain and extraterminal motif (BET) inhibitor;
B is a linker; and
C is 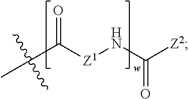
w is 5, 6, 7, 8, 9, or 10;
each Z¹ is independently selected from 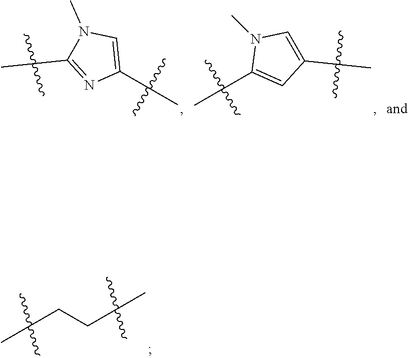
and
Z² is

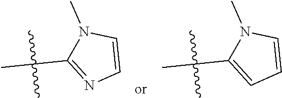

2-3. (canceled)

4. The compound of claim 1, or a pharmaceutically acceptable salt thereof, wherein
C is 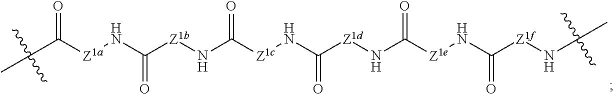
Z^{1a} is 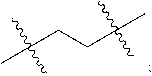 [SMILES: *CC*]
Z^{1b} and Z^{1f} are 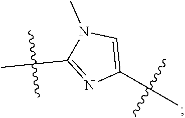 [SMILES: *c1cn(C)c(*)n1]
Z^{1c} is 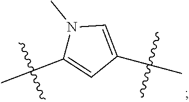 [SMILES: *c1cc(*)n(C)c1]
and
Z^{1d} and Z^{1c} are each independently selected from

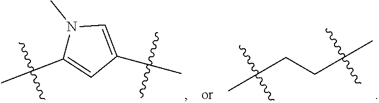

5. The compound of claim 4, or a pharmaceutically acceptable salt thereof, wherein
C further comprises one or more 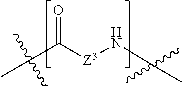
groups, wherein Z³ is selected from

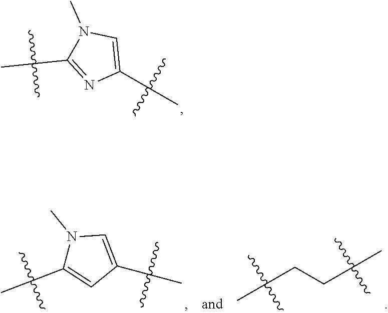

6. The compound of claim 1, or a pharmaceutically acceptable salt thereof, wherein C is selected from the group consisting of:

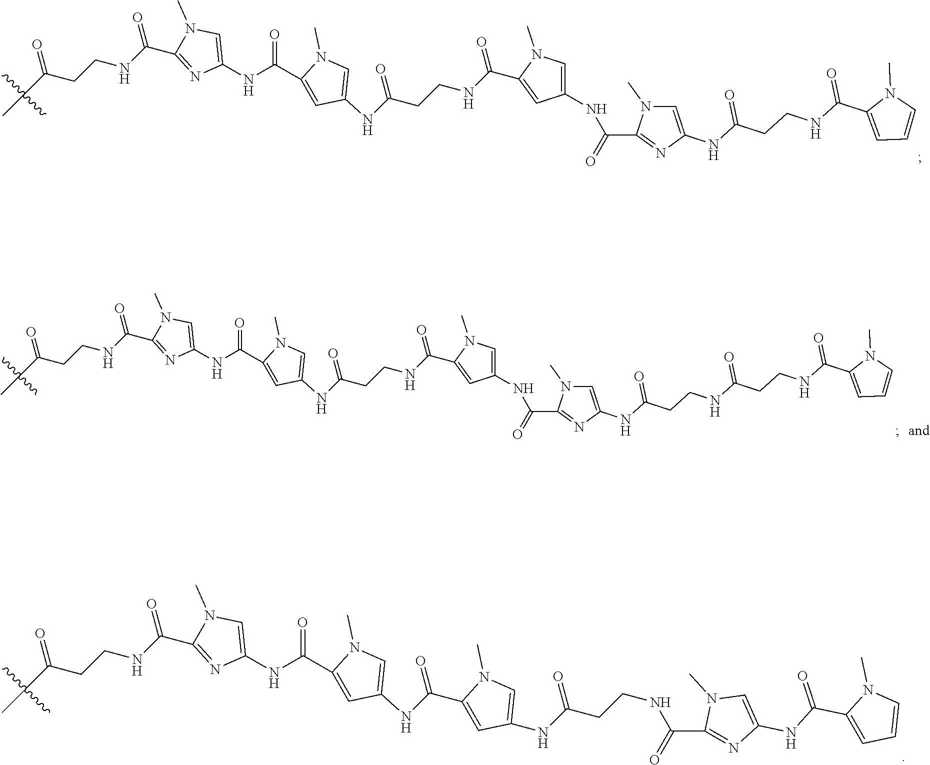 [SMILES: *C(=O)CCNC(=O)c1nc(NC(=O)c2cc(NC(=O)CCNC(=O)c3cc(NC(=O)c4nc(NC(=O)CCNC(=O)c5cccn5C)cn4C)cn3C)cn2C)cn1C ; *C(=O)CCNC(=O)c1nc(NC(=O)c2cc(NC(=O)CCNC(=O)c3cc(NC(=O)c4nc(NC(=O)CCNC(=O)CCNC(=O)c5cccn5C)cn4C)cn3C)cn2C)cn1C ; *C(=O)CCNC(=O)c1nc(NC(=O)c2cc(NC(=O)c3cc(NC(=O)CCNC(=O)c4nc(NC(=O)c5cccn5C)cn4C)cn3C)cn2C)cn1C]

7. (canceled)

8. The compound of claim 1, or a pharmaceutically acceptable salt thereof, wherein A is a group of formula (i): 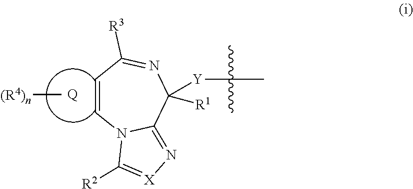
wherein:
Q is a monocyclic 5- or 6-membered heteroaryl having 1, 2, 3, or 4 heteroatoms independently selected from N, O, and S, or phenyl;
R¹ is hydrogen, halogen, or C₁-C₆ alkyl;
R² is hydrogen, C₁-C₆ alkyl, hydroxy-C₁-C₆-alkyl, amino-C₁-C₆-alkyl, C₁-C₆ alkoxy-C₁-C₆-alkyl, halo-C₁-C₆-alkyl, hydroxy, C₁-C₆-alkoxy, or —COO—R³;
R³ is hydrogen, C₁-C₆ alkyl, C₄-C₆ cycloalkyl, C₄-C₆ heterocyclyl, C₄-C₁₀ aryl, or C₄-C₁₀ heteroaryl, wherein each alkyl, cycloalkyl, heterocyclyl, aryl, or heteroaryl is optionally substituted with 1, 2, 3, 4, or 5 substituents independently selected from halo, C₁-C₆ alkyl, C₄-C₆ cycloalkyl, and C₁-C₄ haloalkyl;
n is 1, 2, or 3;
each R⁴ is independently selected from hydrogen, C₁-C₆ alkyl, halo-C₁-C₆-alkyl, C₄-C₆ cycloalkyl, C₄-C₆ heterocyclyl, C₄-C₁₀ aryl, and C₄-C₁₀ heteroaryl, wherein each cycloalkyl, heterocyclyl, aryl, or heteroaryl is optionally substituted; or any two R₄ are taken together with the atoms to which they are attached to form an optionally substituted 5- or 6-membered ring;
X is N or CR⁵;
R⁵ is hydrogen, C₁-C₆ alkyl, C₄-C₆ cycloalkyl, C₄-C₆ heterocyclyl, C₄-C₁₀ aryl, or C₄-C₁₀ heteroaryl;
Y is —C₁-C₆ alkylene-Z— wherein Z is a bond, —C(O)O—, —C(O)—, —S(O)₂—, or —NR⁶—; and
R⁶ is hydrogen or C₁-C₆ alkyl.

9. The compound of claim 8, or a pharmaceutically acceptable salt thereof, wherein
A is a group of formula (ia): 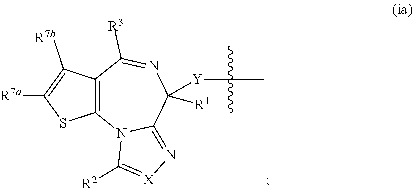
and
R^{7a} and R^{7b} are independently selected from hydrogen, C₁-C₆ alkyl, halo-C₁-C₆-alkyl, C₄-C₆ cycloalkyl, C₄-C₆ heterocyclyl, C₄-C₁₀ aryl, or C₄-C₁₀ heteroaryl.

10. The compound of claim 8, or a pharmaceutically acceptable salt thereof, wherein X is N.

11. (canceled)

12. The compound of claim 8, or a pharmaceutically acceptable salt thereof, wherein Y is —CH₂—C(O)—.

13. (canceled)

14. The compound of claim 8, or a pharmaceutically acceptable salt thereof, wherein R¹ is hydrogen.

15. The compound of claim 8, or a pharmaceutically acceptable salt thereof, wherein R² is hydrogen or C₁-C₆ alkyl.

16. (canceled)

17. The compound of claim 8, or a pharmaceutically acceptable salt thereof, wherein R² is methyl.

18. The compound of claim 8, or a pharmaceutically acceptable salt thereof, wherein R³ is phenyl, optionally substituted with 1, 2, 3, 4, or 5 substituents independently selected from halo, C₁-C₆ alkyl, C₄-C₆ cycloalkyl, C₁-C₄ haloalkyl.

19. The compound of claim 8, or a pharmaceutically acceptable salt thereof, wherein R³ is 4-chlorophenyl.

20-21. (canceled)

22. The compound of claim 9, or a pharmaceutically acceptable salt thereof, wherein both of R^{7a} and R^{7b} are methyl.

23. The compound of claim 8, or a pharmaceutically acceptable salt thereof, wherein the group of formula (i) is:

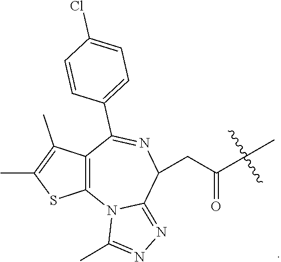 [SMILES: *C(=O)CC1N=C(c2ccc(Cl)cc2)c2c(sc(C)c2C)-n2c(C)nnc21]

24-25. (canceled)

26. The compound of claim 1, or a pharmaceutically acceptable salt thereof, wherein
B is —NR′—(CH₂CH₂O)ₘ—(CH₂)ₚ—C(O)NR′—(CH₂)_{q}—NR′—(CH₂)ᵣ—NR′—;
m, p, q, and r are each independently selected from 1, 2, 3, 4, 5, 6, 7, 8, 9, or 10; and
each R′ is selected from hydrogen or C₁-C₆ alkyl.

27. The compound of claim 1, or a pharmaceutically acceptable salt thereof, wherein B is —NH—(CH₂CH₂O)₆—(CH₂)₂—C(O)NH—(CH₂)₃—N(CH₃)—(CH₂)₃—NH—.

28. The compound of claim 1, wherein the compound is selected from: 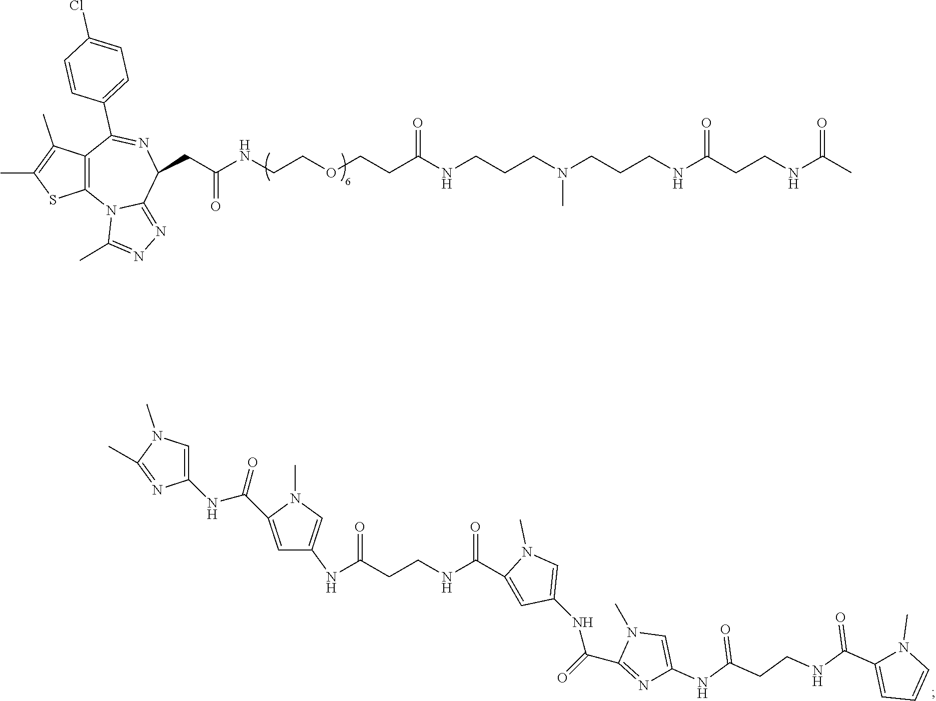 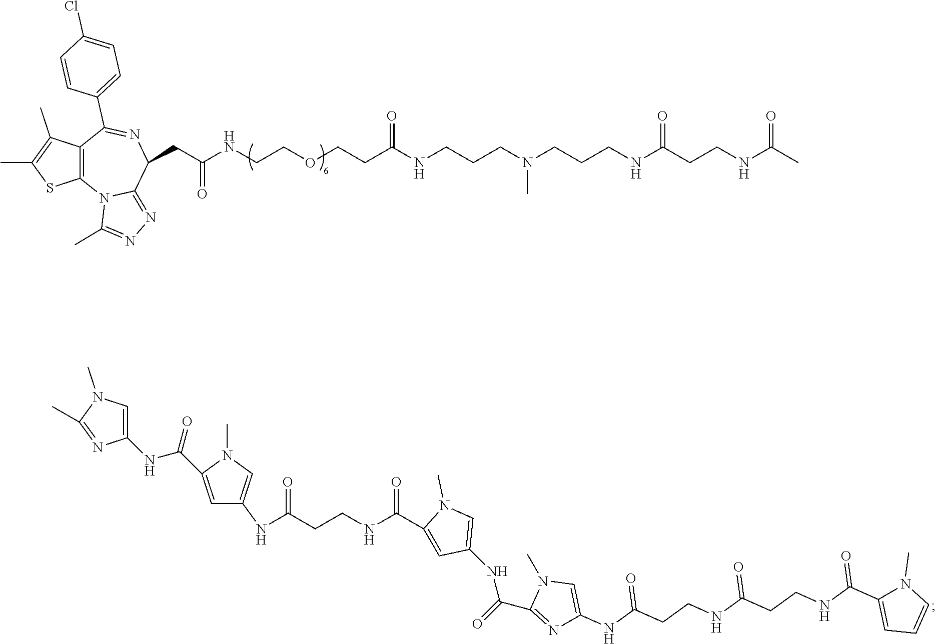 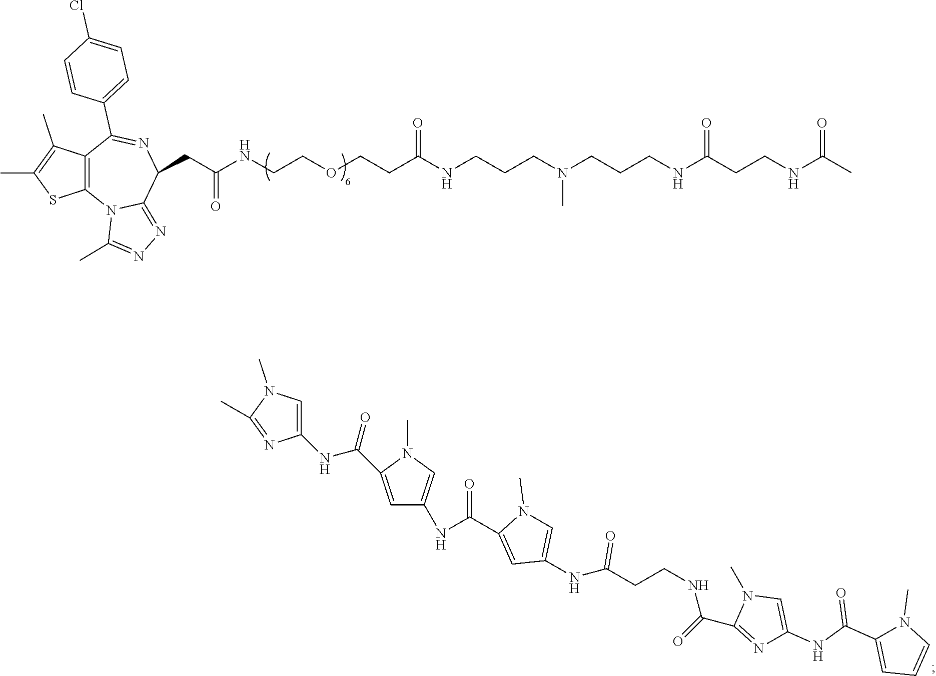
and pharmaceutically acceptable salts thereof.

29. A pharmaceutical composition comprising an effective amount of a compound of claim 1, or a pharmaceutically acceptable salt thereof, and a pharmaceutically acceptable carrier.

30. A method of treating a disease or disorder in a subject in need thereof, comprising administering to the subject an effective amount of a compound of claim 1, or a pharmaceutically acceptable salt thereof.

31-38. (canceled)
